(19) United States
(12) Patent Application Publication
(10) Pub. No.: US 2025/0251662 A1
(43) Pub. Date: Aug. 7, 2025

(54) RADICAL SCAVENGER ADDITIVES FOR METAL OXIDE BASED RESISTS AND PRECURSOR SOLUTIONS

(71) Applicants: Inpria Corporation, Corvallis, OR (US); JSR CORPORATION, Tokyo (JP)

(72) Inventors: Fabian Felix Eberle, Corvallis, OR (US); Amy M. Jystad, Corvallis, OR (US); Brian J. Cardineau, Corvallis, OR (US); Kai Jiang, Corvallis, OR (US); Ken Maruyama, Mie (JP); Seitarou Hattori, Mei (JP); Kazuki Kasahara, Corvallis, OR (US); Colin T. Carver, Corvallis, OR (US); Robert E. Jilek, Philomath, OR (US); Peter De Schepper, Wijnegem (BE)

(21) Appl. No.: 19/042,239

(22) Filed: Jan. 31, 2025

Related U.S. Application Data
(60) Provisional application No. 63/627,974, filed on Feb. 1, 2024.
(60) Provisional application No. 63/648,810, filed on May 17, 2024.

(51) Int. Cl. G03F 7/004; C07C 43/23

(52) U.S. Cl. CPC G03F 7/0045; C07C 43/23; G03F 7/0048

(57) ABSTRACT

An organometallic precursor solution comprising an organic solvent, a radiation sensitive organometallic precursor composition with hydrolysable metal ligands, and a radical scavenger additive is described. The radical scavenger additive or a blend of radical scavenger additives can provide for improvements to the stability of an organometallic precursor solution, such as improvements to storage stability, shelf-life, and/or batch-to-batch reproducibility through mitigation of the effects of reactive compounds in the environment, such as oxygen. A structure having a substrate, a radiation patternable organometallic coating composition, and a radical scavenging additive is also described. The radical scavenger additive or a blend thereof can result in patterning improvements, such as by improving coating quality and reducing patterning variability. Methods of using a radical scavenging additive in the formation of a structure comprising a radiation patternable organometallic film are described. Methods of producing a container of a stabilized organometallic precursor composition are also described.

### CROSS REFERENCE TO RELATED APPLICATIONS

This application claims priority to copending U.S. provisional patent application 63/627,974 filed on Feb. 1, 2024 to Cardineau et al., entitled “Radical Trapping Additives for Metal Oxide Resists,” and to copending U.S. provisional patent application 63/648,810 filed May 17, 2024 to Jilek et al., entitled “Additives for Improving Environmental Stability of Metal Oxide Resists and Precursor Solutions,” both of which are incorporated herein by reference.

### FIELD OF THE INVENTION

The invention relates to organometallic, in particular organotin, patterning compositions with additives, such as antioxidants and/or radical scavengers, to improve uniformity of process conditions. Specifically, the additives can particularly improve the stability of metal oxide precursor solutions and/or metal oxide resist films through mitigation of the effects of reactive compounds in the environment, such as oxygen. The improved stability can result in improvements in coating quality, reproducibility, and mitigation of aging effects of the resist films, for example.

### BACKGROUND OF THE INVENTION

Semiconductor lithography is a complex and critical technology used to fabricate myriad and diverse devices that have dominated and transformed the modern world beginning in the 20^{th} century. The semiconductor lithographic process is generally an iterative process involving repeated steps of deposition, patterning, and etching of many layers and materials to form the desired devices. As technology advances and new, increasing demands and requirements are placed upon each generation of devices, the need to develop processes and materials that are able to meet these requirements increases. One of the critical materials used in the semiconductor lithographic process is the photoresist in which an initial pattern is formed by exposure to radiation and is then subsequently transferred into the underlying substrate.

Organometallic photoresists have been shown to be promising materials for use in current and next-generation semiconductor lithography processing due to their ability to form high-resolution, high etch resistance, and high-fidelity patterns. These organometallic systems generally operate through radiation exposure-mediated formation of condensed oxide networks that drive contrast between irradiated (i.e., exposed) and non-irradiated (i.e., unexposed) regions of the material. A development process can then be used that can selectively remove the irradiated or the non-irradiated material to realize a physical pattern of material based on a latent image formed by the pattern of radiation. The presence of certain airborne and/or environmental contaminants during semiconductor device manufacturing, such as lithographic processing, can have undesirable and significant effects on the performance of metal oxide resist (MOR) materials, including changes in sensitivity, line width roughness (LWR), resolution, and defectivity.

### SUMMARY OF THE INVENTION

One aspect of the invention pertains to a precursor solution comprising organic solvent, a radiation sensitive organometallic precursor composition with hydrolysable metal ligands, and a radical scavenger additive.

Another aspect of the invention pertains to a structure comprising a substrate having a surface and a radiation patternable coating along the substrate surface comprising a radiation sensitive metal oxide based, organometallic composition and a radical scavenging additive.

Another aspect of the invention pertains to a method of forming a radiation patternable metal oxide-based coating on a substrate surface. The method comprises exposing the substrate surface to an organometallic precursor composition with hydrolysable ligands, exposing the substrate surface to vapor of a radical scavenging additive, and forming a radiation sensitive organometallic film associated with the radical scavenging additive. The exposure to the radical scavenging additive is before, during, after or any combination thereof with respect to exposure to the organometallic precursor composition.

In a further aspect, the invention pertains to a method of producing a container of radiation sensitive organometallic precursor composition. The method comprises forming a stabilized solution comprising dissolved organometallic precursor composition and a dissolved selected quantity of radical scavenging additive and sealing the stabilized organometallic precursor solution in a container for distribution.

### BRIEF DESCRIPTION OF THE DRAWING

FIG. 1 is a process flow diagram illustrating aspects of the experimental protocol of Example 3.

### DETAILED DESCRIPTION OF THE INVENTION

The addition of radical scavengers to organo metal oxide resist (MOR) compositions can alleviate the undesired effects of certain environmental contaminants during lithographic processing. Unless specifically indicated otherwise, a reference to MOR compositions covers precursors, resulting radiation sensitive films or both. MOR compositions of particular interest are organo tin compositions, which are generally formed from precursors having hydrolysable ligands with a non-hydrolysable ligand forming a C—Sn bond. In particular, the patterning of organometallic photoresists can be improved in some circumstances by the presence of the additive(s) in the photoresist film and/or in the precursor solution. The additives can inhibit the formation of or prevent reactions with reactive species, such as peroxides or radicals, and can thus alleviate certain undesired effects of environment contaminants during lithographic processing, generally avoiding side reactions that can reduce patterning contrast. Generally, the radical scavenger additives, which in some embodiments may be characterized as radical trapping agents and/or antioxidants, can stabilize the organometallic compositions towards reaction with reactive species, such as radicals and/or radical-generating environmental contaminants, such as oxygen, reactive oxygen species, reactive nitrogen species, reactive sulfur species, or the like, which generally results in the formation of a less reactive product that is less prone to react with the photoresist. The improvements resulting from the additives can be particularly effective in the context of reducing photoresist processing variability, such that temporal changes in resist performance in the presence of reactive species can be lessened. The radical scavenging additives can be useful, for example, to mitigate aging or delay effects that reduce shelf life, reduce patterning variability, improve coating quality of the metal oxide resists, and improve batch-to-batch reproducibility.

The ability of organometallic photoresist compositions to facilitate extremely high resolution radiation based patterning and lithography is complicated by a correspondingly high sensitivity to contaminants that can result in variability of coating solutions used to form photoresist films and/or variability in the photoresist films themselves. For example, the presence of certain airborne and/or environmental contaminants during various stages of semiconductor device manufacturing, such as preparation and handling of precursor solutions and photoresist film processing, can have undesirable and significant effects on the performance of metal oxide resist (MOR) materials, including changes in sensitivity, contrast, line width roughness (LWR), resolution, and defectivity. It is therefore desirable to design organometallic photoresist compositions that are more resistant to environmental contaminants.

Metal oxide (organic modified) based patterning compositions have taken a leading role in high resolution patterning, especially for extreme ultraviolet (EUV) patterning. These resist materials provide challenges in achieving the full potential with respect to high resolution patterning capability. Organometallic photoresist compositions are described herein that comprise additives to improve patterning performance and consistency. The additives or portions thereof may or may not persist in the resulting radiation sensitive films. In general, the organometallic photoresists can be deposited from solution or with vapor deposition, and for stabilization of the radiation sensitive films. Appropriate additives can correspondingly be deposited with either approach. If the additives do not persist in the radiation sensitive films, the additives can be effective to provide storage stability for precursor solutions. A blend of additives can be used in which a portion of the additives persist in the radiation sensitive films while other additives are removed during deposition and/or heating.

While Applicant has pioneered and made great advances with organotin based resists, other metal based resist formulations have been explored. Applicant's earlier work involved radiation sensitive peroxide ligands with Hf or Zr ions, see for example, in U.S. Pat. No. 9,176,377B2 to Stowers et al., entitled “Patterned Inorganic Layers, Radiation Based Patterning Compositions and Corresponding Methods,” incorporated herein by reference. Related resist compounds are discussed in published U.S. patent application 2013/0224652A1 to Bass et al., entitled “Metal Peroxo Compounds With Organic Co-ligands for Electron Beam, Deep UV and Extreme UV Photoresist Applications,” incorporated herein by reference. Other resists are described in published U.S. patent application 2009/0155546A1 to Yamashita et al., entitled “Film-Forming Composition, Method for Pattern Formation, and Three-Dimensional Mold,” and U.S. Pat. No. 6,566,276 to Maloney et al., entitled “Method of Making Electronic Materials,” both of which are incorporated herein by reference. See also, published U.S. patent application 2012/0315451 to Malik et al., entitled “Metal-Containing Compositions and Methods of Making Same,” incorporated herein by reference. However, the rest of the discussion focused on organotin-metal oxide based patterning compositions in the context of references to more generally applicability for organometallic resists.

When present in the MOR compositions, the radical scavenger additives can stabilize the material against reaction with radical-generating contaminants, such as reactive oxygen species (ROS, e.g., superoxide, peroxides, hydroperoxide, hydroxy radicals, etc.), reactive nitrogen species (RNS, e.g., NOx compounds such as NO, NO₂, and related compounds), reactive sulfur species (RSS, e.g., SO₂), other radical generating species, and mixtures thereof. These species may be air borne or introduced as contaminants from various components of the resist formulations, such as a solvent. Additives blended with the precursor solutions can competitively react with reactive species and thereby reduce the likelihood of these reactive species reacting with the organometallic compositions in the precursor solutions as well as a resulting film. The reactions of the radical scavenging additives with reactive contaminants generally form products that, while also reactive, are less reactive and less likely to result in damaging reactions with the photoresist. For vapor deposited MOR compositions, additives can be introduced in the vapor phase. The stability of the MOR films can be enhanced such that temporal changes in resist performance in the presence of radical-inducing species can be lessened.

In practice, the stabilization mechanism may not be elucidated, but the presence of additives can be correlated with any observed patterning improvements. The radical scavenging additives can be selected for their ability to prevent peroxide formation, prevent free radical formation, scavenge radicals, or a combination thereof, and the mode of operation of the additives may not be known or particularly relevant. The net effect of radicals may be damage to the resist material that degrades patterning. While not wanting to be limited by theory, this damage to the photoresist may be associated with cleavage or other damage to the organo ligand bound to the tin atom. As used herein, radical scavenging refers to any action to limit or remove damage from radicals or other highly reactive species degrading patterning. In some embodiments, the additives can be additionally selected for case of removal of any unreacted additive and/or reaction products of the additive from the precursor solution or from a coated film at desired timing in the processing. The additives are generally selected to provide improved consistency of patterning results, which may involve avoiding the additives' effects on patterning performance in the absence of contaminants while also effectively reducing the effects of such species that would otherwise negatively interact in some way with the photoresist material.

As noted above, radical scavenging additives can be effective to improve precursor solution stability, which may increase shelf life, and/or improve performance of the film following coating. If the primary effect is to improve precursor solution stability, the additive can be selected to be sufficiently volatile that it is substantially removed during deposition or a relatively mild post application (pre-exposure) bake. Other additives may persist all the way through processing or are removed during a post-exposure bake and/or a post-development bake. For additives that are provided in a vapor deposition step for forming the radiation patternable film, the additive generally persists then through processing or is removed during a post-exposure bake or a post-development bake. Vapor deposited additives should have sufficient vapor pressure to allow for forming volatile species for deposition onto the substrate surface simultaneously or sequentially with the photoresist. Thus, vapor deposited additives should have a boiling point low enough to allow for vapor formation, but high enough to allow for condensation onto the substrate and optionally retention through a post application bake step, which can be useful even for vapor deposited photoresist precursors.

The irradiation process, especially with EUV radiation introduces considerable energy that can result in radical formation. To the extent that these radicals can migrate from the targeted irradiation zone, pattern blurring can occur. Radical scavenging additives present in the films during patterning can be used to reduce pattern blurring. Thus, it can be desirable to maintain radical scavenging additives in the film after precursor solution deposition or to introduce radical scavenging additives in the film through vapor deposition. If desired, a radical scavenging additive can be provided in a precursor solution that is volatilized upon delivery while a second radical scavenging additive can remain in the resulting film through irradiation.

The MOR compositions can generally be stable in the presence of the radical scavenger compounds. When present in the MOR compositions, the radical scavenger compounds can preferentially react with airborne contaminant species that would otherwise react with the organo metal oxide composition. By including the radical scavenger compounds in the organo metal oxide resist precursor compositions, reactions with radical-generating species and/or radicals that can lead to undesired and/or unpredictable changes in patterning performance can be mitigated. As demonstrated in the Examples, use of the radical scavenging additives can significantly improve patterning uniformity upon appropriate resist storage times.

Organometallic photoresist compositions (organic modified-metal oxide resists) are a class of materials pioneered by Applicant and used in the field of semiconductor manufacturing and photolithography. Like conventional polymer-based resists, they serve as a radiation-patternable layer that can be exposed to a pattern of radiation and selectively developed to create a physical mask on a substrate that corresponds to the pattern of radiation, and which then can guide the subsequent etching and deposition processes used to fabricate semiconductor devices. Metal oxide resists are typically composed of ligand-stabilized metal oxides, such as those with organo ligand-metal bonds, that can change their solubility properties when exposed to radiation, such as UV light, electron beams, or extreme ultraviolet (EUV) light.

Applicant has described the use of additives, such as a photoacid generator and/or a quenching agent, to reduce blurring of a patterned organometallic resist, see, for example, published U.S. patent application 2024/0085785 to Kasahara et al., entitled “Additives for Metal Oxide Photoresists, Positive Tone Development With Additives, and Double Bake Double Develop Processing,” incorporated herein by reference. The patterning compositions described herein provide mitigation of the effects of environmental contaminants, such as by limiting the uncontrolled formation of peroxides and/or free radical species in the compositions that can occur due to exposure of the solutions and films to various species (for example, oxygen and/or reactive oxygen species) during storage, handling, or processing. U.S. provisional patent application 63/638,615 (“the '615 application”) to Boutilier et al., entitled “Peroxide-Stabilized Organotin Photoresist Compositions and Patterning,” incorporated herein by reference, describes a different approach to peroxides in an organometallic photoresist composition. In particular, the '615 application describes adding controlled amounts of specific peroxide compounds to organotin photoresist compositions in order to enhance their solution stability with respect to hydrolysis/condensation and radiation sensitivity. For example, the '615 application describes mixing an aqueous hydrogen peroxide solution with an organotin photoresist solution to achieve a selected concentration or ratio between peroxide and tin (Sn). Through the use of the additives described herein, further improvements to organometallic photoresist solutions and films are achieved by mitigating uncontrolled reactions, such as with O₂, peroxides, other ROS, and/or radicals, that are driven by the presence of reactive compounds in the environment and that can lead to patterning variability.

Organotin compounds, particularly those based on monoalkyltin trialkoxide (RSn(OR′)₃) and monoalkyltin triamide (RSn(NR′₂)₃) precursor compounds, are particularly useful MOR compositions for EUV lithography. The use of alkyltin compounds in high performance radiation-based patterning compositions is described, for example, in U.S. Pat. No. 9,310,684 to Meyers et al. (hereinafter the '684 patent), entitled “Organometallic Solution Based High Resolution Patterning Compositions,” incorporated herein by reference. Refinements of these organometallic compositions for patterning are described in U.S. Pat. No. 10,642,153 to Meyers et al., entitled “Organometallic Solution Based High Resolution Patterning Compositions and Corresponding Methods,” and 10,228,618 to Meyers et al. (hereinafter the '618 patent), entitled “Organotin Oxide Hydroxide Patterning Compositions, Precursors, and Patterning,” both of which are incorporated herein by reference.

The organotin precursor compositions comprise a group that can be hydrolyzed with water or other suitable reagent under appropriate conditions to form monoorgano tin oxo-hydroxo patterning compositions, which, when fully hydrolyzed, can be represented by the formula RSnO_{(1.5-(x/2))}(OH)ₓ where 0<x≤3. It can be convenient to perform the hydrolysis to form the oxo-hydroxo compositions in situ, such as during deposition and/or following initial coating formation. While alkyl tin triamides and alkyl tin triacetylides described, for example, in the above-referenced '618 patent, can be used under hydrolyzing conditions for forming radiation sensitive coatings for patterning, it can be desirable to use alkyl tin trialkoxides as part of solution-based film-forming compositions. As used in this art, ligands bound to a tin atom with a C—Sn bond can be equivalently referred to as alkyl ligands, hydrocarbyl ligands or organo ligands with the understanding that the ligand can comprise heteroatoms, and unsaturated bonds while providing a suitable C—Sn bond.

Monoalkyl tin precursor compositions can generally be represented by the formula RSnL₃, where R is an alkyl (organo) group having a radiation-sensitive Sn—C bond and L is a hydrolysable ligand. For processing to form radiation patternable coatings, L is generally hydrolysed before, during (e.g., in-situ) deposition, and/or during solvent removal to result in a coating comprising a polymeric organotin oxo-hydroxo composition on a substrate wherein the Sn—R bonds remain substantially intact. As a result, a radiation patternable coating having radiation-sensitive Sn—R bonds can be realized. While not wanting to be limited by theory, it is generally believed that the radiation patternable coating has an oxo-hydroxo network modulated by at least the non-bridging organo ligands and potentially other components, while irradiation and condensation can ultimately drive the material more into a metal oxide structure. Generally, the radiation patternable coating material formed from the organotin precursors has Sn—C, Sn—OH and Sn—O—Sn bonds.

Processing of the organotin precursor compositions to afford organotin oxo-hydroxo coatings generally involves hydrolysis of the RSnL₃ composition(s) to afford the related organotin oxo-hydroxo composition(s). Hydrolysis can be performed prior to the deposition process to yield soluble organotin oxo-hydroxo species (i.e., clusters, oligomeric species, etc.). These soluble organotin oxo-hydroxo species can then be dissolved and/or dispersed into a suitable solvent to form an organotin photoresist solution that can then be used to form radiation-patternable organotin oxo-hydroxo coatings. Alternatively, the organotin precursor compositions can be directly dissolved in a suitable solvent to form a photoresist solution that can then be used to form radiation-patternable organotin oxo-hydroxo coatings. The resulting coatings generally comprise Sn—C, Sn—OH and Sn—O—Sn bonds, in which the Sn—C bonds are radiation sensitive, which evidence suggests results in cleavage under irradiation and/or subsequent heating. The organotin precursor compositions can also be hydrolysed in-situ with water during the substrate coating process, such as during vapor deposition. Various processing options are described further in the '684 and '618 patents referenced above.

For organotin photoresist compositions wherein the organotin precursor(s) are dissolved into a solvent for spin-coating, organotin trialkoxides (RSnL₃, L=OR′) can be desirable for use over other RSnL₃ compositions (e.g, organotin triamides, L=NR′₂). Some advantages to organotin trialkoxide compositions are, for example, the production of more benign side-products, e.g., alcohols, that are relatively innocuous compared to the production of gaseous products (e.g., amines) which may cause contamination concerns, environmental health and safety concerns, and/or similar concerns within the wafer track and/or wafer fab. While organotin triamides are known to be useful as precursors in vapor-based deposition methods (as described in the '618 patent), organotin trialkoxides also possess appreciable vapor pressures and low melting points which make them attractive compounds for use in vapor deposition methods to prepare radiation-patternable coatings. For the hydrolysable ligands, the art also recognizes that a reference to an alkyl group is similarly used interchangeably with an organo groups that can include heteroatoms and or unsaturated bonds.

The organotin compositions can generally comprise one or more distinct compounds each represented by the formula RₙSnL₄₋ₙ, where n=1, 2, or 3 and where R forms a carbon-tin bond that can optionally comprise heteroatoms, which are not carbon or hydrogen. In the cases where multiple tin species are present in the organotin composition as a blend, the overall formula of the blend can generally be represented by the averaged formula RₙSnL₄₋ₙ, where 0.5<n<2. As noted above, for convenience as well as consistency in the art, R can be interchangeably referred to as an alkyl ligand, organo ligand or hydrocarbyl ligand. The hydrolysable ligand, L, generally includes ligands that can be hydrolyzed with water, such as alkoxides (hydrocarbyl oxide, OR′), acetylides (C≡CR′), carboxylates (O₂CR′), or amides (NR′₂). In some embodiments, R′ is a linear, branched, cyclic, or aromatic hydrocarbyl group having from 1 to 10 carbon atoms. In some embodiments, R′ can include methyl, ethyl, propyl, propyl, butyl, pentyl, silyl, any of their respective isomers, and combinations thereof. In some embodiments, branched alkyl ligands can be desirable for some patterning compositions where the compound can be represented generally as R¹R²R³CSn O_{(2-(z/2)-(x/2))}(OH)ₓ, where R¹, R² and R³ are independently hydrogen or an alkyl group with 1-10 carbon atoms. Similarly, this representation of alkyl ligand R is similarly applicable to the other embodiments generally with R¹R²R³CSn(L)₃, with L corresponding to hydrolysable ligands, such as alkoxide (hydrocarbyl oxide), acetylide, carboxylate, or amide moieties. In some embodiments, R¹ and R² can form a cyclic alkyl moiety, and R³ may also join the other groups in a cyclic moiety. Suitable branched alkyl ligands can be, for example, isopropyl (R¹ and R² are methyl and R³ is hydrogen), tert-butyl (R¹, R² and R³ are methyl), tert-amyl (R¹ and R² are methyl and R³ is —CH₂CH₃), sec-butyl (R¹ is methyl, R² is —CH₂CH₃, and R³ is hydrogen), neopentyl (R¹ and R² are hydrogen, and R³ is —C(CH₃)₃), cyclohexyl, cyclopentyl, cyclobutyl, and cyclopropyl. Examples of suitable cyclic groups include, for example, 1-adamantyl (—C(CH₂)₃(CH)₃(CH₂)₃ or tricyclo(3.3.1.13,7) decane bonded to the metal at a tertiary carbon) and 2-adamantyl (—CH(CH)₂(CH₂)₄(CH)₂(CH₂) or tricyclo(3.3.1.13,7) decane bonded to the metal at a secondary carbon). In other embodiments, hydrocarbyl groups may include aryl or alkenyl groups, for example, benzyl or allyl, or alkynyl groups. In other embodiments, the hydrocarbyl ligand R may include any group consisting solely of C and H and containing 1-31 carbon atoms. In summary, some examples of suitable alkyl groups bonded to tin include, for example, linear or branched alkyl (i-Pr ((CH₃)₂CH—), t-Bu ((CH₃)₃C—), Me (CH₃—), n-Bu (CH₃CH₂CH₂CH₂—)), cyclo-alkyl (cyclo-propyl, cyclo-butyl, cyclo-pentyl), olefinic (alkenyl, aryl, allylic), or alkynyl groups, or combinations thereof. In further embodiments, suitable R groups may include hydrocarbyl groups substituted with hetero-atom functional groups including cyano, thio, silyl (and germanium analogs), ether, keto, ester, or halogenated groups or combinations thereof, such as one or more fluorine atoms and/or on or more iodine atoms. As is conventional in this art, the hydrocarbyl group can be referred to as an alkyl group even though the group can have unsaturated bonds, aryl groups, heteroatoms, and so forth. Recent synthesis efforts have resulted in commercially practical approaches to form compositions with a greater range of R ligands, as described in published U.S. patent application 2024/0199658 to Jilek et al., entitled “Direct Synthesis of Organotin Alkoxides,” U.S. Pat. No. 10,787,466 to Edson et al., entitled “Monoalkyltin compositions with low polyalkyl tin contamination, their compositions and methods”, and U.S. patent application Ser. No. 17/410,316 to Edson et al., entitled “Methods to produce organotin compositions with convenient ligand providing reactants”, all of which are incorporated herein by reference. As described in the aforementioned references, various heteroatoms can be introduced into the ligands, including F, I, N and O atoms.

In some embodiments the precursor compositions comprise a mixture of R—Sn moieties with hydrocarbyl ligands and inorganic SnL₄ compounds, wherein L is defined within the scope of L as in the RSnL₃ compositions above. In some embodiments, SnL₄ is a tin tetraalkoxide, Sn(OR′)₄, or tin tetraamide, Sn(NR′₂)₄, wherein R′ is defined within the scope of R′ as in the RSnL₃ compositions above. In embodiments wherein the inorganic SnL₄ compound is included in the organotin photoresist composition, the SnL₄ can comprise no more than about 30 mol. % of the total moles Sn in the photoresist composition, no more than about 20 mol. % in some embodiments, and no more than about 15 mol. % in further embodiments. In some embodiments, the SnL₄ compound can comprise, with any of the upper limit values above, at least about 0.5 mole percent of the total moles Sn, in some embodiments at least about 5 mole percent, and in further embodiments at least about 10 mole percent of the total moles Sn. A person of ordinary skill in the art will recognize that additional ranges of mol. % SnL₄ within the explicit ranges above are contemplated and are within the present disclosure.

As described herein, the radical scavenging additives can generally be added to the organotin resist compositions to form radical-stabilized resist compositions. The radical scavengers can generally comprise compounds capable of neutralizing and/or counteracting radicals or radical-generating species through reactions that result in less reactive products. The radical scavenging additives can be added to the organotin resist compositions to mitigate the effects of environmental contaminants during storage, handling, and/or semiconductor processing. These compounds are capable of neutralizing reactive radical or radical-generating species that could otherwise react with the resist material and lead to degradation of its performance.

Radical-generating contaminants or the like are generally compounds that have the potential to interact with the photoresist film and/or other species to result in the formation of radicals or similar highly reactive species. The radical-generating contaminants can react directly on the photoresist composition to generate radicals or they may first generate radicals prior to interaction with the photoresist which then can interact with the resist. In any case, the radicals can initiate a series of chemical changes within the photoresist that are generally undesirable. The radical-induced chemical changes can alter the properties of the photoresist and can potentially compromise its lithographic performance.

There are numerous types of compounds that can act as these detrimental radical-generating contaminants. Some of the non-limiting examples include reactive oxygen species (ROS), such as superoxide, hydroperoxide, and hydroxyl radicals, and nitrogen oxides (NOx), including NO, NO₂, and other related nitrogen oxides. These compounds are particularly problematic as they are capable of generating radicals that can propagate through the photoresist, causing a cascade of chemical changes.

The radical-generating contaminants can be introduced into the processing environment in a variety of ways, for example through the inadvertent introduction of contaminated airflows during photoresist formulation and/or lithographic processing. Introduction of radical-generating contaminants can occur due to a variety of factors, such as inadequate filtration systems, leaks in the processing equipment, or using contaminated materials during the manufacturing process. Regardless of the source, the presence of these contaminants during the processing of the metal oxide photoresist can lead to a degradation in the performance of the photoresist, underscoring the imperative for effective mitigation strategies such as through the presence of radical-scavenger agents in the photoresist composition. Results in the examples suggest radical-generating contaminants can result from contact with air or due to contaminants in precursor solutions even when formed using highly purified compounds.

While not wanting to be limited to be limited by theory, it is believed that exposure of organotin resists to certain environment contaminants, such as radical-generating airborne contaminants, can lead to undesired cleavage of the Sn—C bond via complex radical chemistry and thus lead to unpredictable changes in resist performance. In general, radical-generating contaminant species can lead to generation and/or initiation of radicals in the organotin resist film. The radicals can react with the organotin matrix through a variety of mechanisms and pathways which can lead to chemical changes in the material, the formation of insoluble or less soluble products, or similar changes that can lead to changes in resist performance. Propagation of radical reactions may also occur such that small concentrations of radical-generating contaminants and/or radicals can lead to significant changes in resist performance on relatively short timescales. Therefore, the addition of a suitable radical scavenger additives to the resist composition can mitigate these reactions. The scavengers can preferentially react with the radical-generating contaminants and/or the radicals formed during the process, thereby preventing the initiation and propagation of the chain reactions that lead to resist degradation and thereby stabilizing the resist material and maintaining its performance during the lithographic process.

In some embodiments, the organotin composition comprises a mixture of organotin compounds having different hydrocarbyl (organo) R groups with the same and/or different L groups bound accordingly. In some embodiments, the organotin composition has R ligands comprising a blend of a linear alkyl ligand and a non-linear alkyl ligand. In some embodiments, a blend of a linear alkyl ligand and a non-linear alkyl ligand comprises a branched alkyl group, a cyclo-alkyl group, or an aryl group. In some embodiments, the organotin composition is a mixture of distinct organotin compounds having methyl ligands and t-butyl ligands, respectively. In some embodiments, the precursor compositions comprise a blend of organotin compounds having the same and/or different L groups. In some embodiments, the organotin composition comprises distinct organotin compounds with L ligands comprising a dialkylamide, an alkylsilylamide, an alkyloxide, an alkylacetylide, or a combination thereof. In other embodiments, the organotin composition comprises distinct organotin compounds with L ligands comprising methoxide, ethoxide, propoxide, iso-propoxide, butoxide, iso-butoxide, tert-butoxide, tert-amyloxide, pentoxide, iso-pentoxide, dimethyl amide, diethyl amide, diiso-propyl amide, trimethylsilyl amide, other isomers thereof, or combinations thereof. In some embodiments, the organotin composition further comprises an inorganic SnL₄ compound such as within the amount ranges given above. In embodiments wherein a blend of different organotin compounds are used, any one of the distinct organotin compounds can comprise from about 1 mol. % to about 99 mol. % of the total organotin moles in some embodiments, from about 5 mol. % to about 95 mol. % of the total organotin moles in other embodiments, from about 10 mol. % to about 90 mol. % of the total organotin moles in other embodiments, and from about 15 mol. % to about 85 mol. % of the total organotin moles in further embodiments. In the Examples below, organotin precursor solutions comprising a mixture of two distinct alkyltin tri-tert-amyl alkoxide compounds are demonstrated. As is conventional in this art, the hydrocarbyl group can be referred to as an alkyl group even though the group can have unsaturated bonds, aryl groups, heteroatoms, and so forth or as an organo group with its various stoichiometric possibilities.

While not wanting to be limited to be limited by theory, it is believed that exposure of organotin photoresists to certain environment contaminants, such as radical-generating contaminants, can lead to undesired cleavage of the Sn—C bond via complex radical chemistry and/or redox chemistry and thus lead to unpredictable or uncontrolled changes in resist performance. In general, radical-generating contaminant species can lead to generation and/or initiation of radicals in the organotin photoresist film. The radicals can react with the organotin matrix through a variety of mechanisms and pathways which can lead to chemical changes in the material, to produce products with different solubility properties or with similar property changes that can lead to changes in resist performance. Propagation of initiated radical reactions may also occur such that small concentrations of radical-generating contaminants and/or radicals can lead to significant changes in resist performance on relatively short timescales. Therefore, the addition of suitable radical scavenger additives, such as radical trapping agents and/or antioxidants, to the resist composition can mitigate these reactions. The additives can preferentially react with the radical-generating environmental contaminants and inhibit the formation of ROS or radical species and/or further react with ROS or other radical generating species to inhibit radical formation. The radical quenchers can preferentially react with the radical-generating contaminants and/or the radicals formed during the process, thereby preventing the initiation and propagation of the chain reactions that lead to resist degradation and thereby stabilizing the resist material and maintaining its performance during the lithographic process. Therefore, introduction of suitable radical scavenger additives can improve metal oxide (MO) resist performance and significantly improve consistency of pattern formation from use of the MO resists.

Stability against the effects of radicals and related reactive species can be considered in several different ways. Some aspects manifest themselves in the ability to form reproducibly a good patternable film. Thus, a precursor solution should not have precipitates forming during storage, and coating of the solution should form a film with reproducible characteristics. Ultimately though, uniformity should extend to the patterning. In the Examples below, patterning uniformity is evaluated through maintenance of dose-to-size (DtS) values. Example 2 explores changes in DtS resulting from irradiation and Example 3 explores the effects on DtS from storage of resist solution. In any case, it is generally desirable for the absolute value of DtS changes in circumstances where radicals or related reactive species are generated to be no more than about 10%, in further embodiments no more than about 5%, and in additional embodiments no more than about 3%. A person of ordinary skill in the art will recognize that additional ranges of DtS changes within the explicit ranges above are contemplated and are within the present disclosure.

### Organometallic Precursor Solutions and Radical Scavenging Additives for Solution or Vapor

Precursors can be delivered to a substrate surface using solution deposition or vapor deposition approaches. Precursor solutions generally comprise an organometallic precursor dissolved in an organic solvent, which can have a suitable radical scavenging additive dissolved in the solution. As noted above, organometallic precursors of particular interest are mono-organo tin compositions with hydrolysable ligands. The additives can be selected to be sufficiently soluble for dissolving into the solvent for forming a precursor solution or to have sufficient vapor pressure for vapor deposition. With respect to vapor delivery, some of the radical scavenging agents have sufficient vapor pressure that they can be supplied during vapor delivery of the organometallic precursors or separately for incorporation into the resulting film. As noted in the following section, the radical scavenging additives can be incorporated into the resulting radiation sensitive films. Suitable radical scavenging additives can include, for example, radical trapping agents, antioxidants and the like.

The resist precursor composition can be conveniently specified based on tin ion molar concentration. In general, the resist precursor solution generally comprises from about 0.0025 M to about 1 M tin cation, in some embodiments from about 0.004M to about 0.9M, in further embodiments from about 0.005 M to about 0.75 M, also in some embodiments from about 0.01M to about 1M, and in additional embodiments from about 0.01 M to about 0.5 M tin cation. The desirable concentrations of radical scavenging additive can be expressed as molar ratios relative to the total Sn atom concentration, e.g. moles additive to moles Sn, in the ultimate organotin photoresist precursor solution, or they can be expressed as molarities. In some embodiments, the radical scavenging additive can be present in the organotin photoresist composition in a molar ratio of radical scavenging additive to Sn in a range from a lower limit of about 0.00002, in other embodiments about 0.00005, in further embodiment about 0.000075, in additional embodiments about 0.00001 to an upper limit independently of any one of about 0.5, in some embodiments of about 0.6, in further embodiments of about 0.75 in some embodiments, and in additional embodiments of about 0.8. For vapor deposited additives, the same ratios of additive to tin are applicable. Similarly, a precursor solution can comprise radical scavenging additive at a concentration from about 0.000001M to about 0.4M in further embodiments form about 0.000002M to about 0.3M, in additional embodiments from about 0.0000035M to about 0.25M, in some embodiments from about 0.000004M to about 0.2M, and in further embodiments from about 0.000005M to about 0.15M, or any range with any one of these lower molarity limits with any one of the explicit upper molarity limits. Other ranges for molar ratios of radical scavenging agent to Sn and concentrations of additive in precursors solutions not directly disclosed but within the above ranges are contemplated and are within the scope of the present disclosure.

The radical scavenging additives can generally be included in the organotin photoresist solution compositions by dissolving or mixing an appropriate amount of additive into the photoresist solution. The radical scavenging additives are combined with precursors in a solvent to form precursor solutions for the organotin patterning compositions for delivery to a substrate. Solvents for the organotin precursors are generally suitable for dissolving the radical scavenging additive can be selected accordingly. In some embodiments, the radical scavenging additive can be added directly to an organotin photoresist solution composition in a desired amount. In some embodiments, to facilitate dissolving, the radical scavenging additives can be dissolved into a suitable solvent to form a radical scavenging additive solution prior to mixing the additive solution with the organotin photoresist composition. In these embodiments, the solvent for the additive solution generally is the same as the solvent for precursor solution or is miscible with or soluble in the solvent for the precursor solution.

The radical scavenging solution can generally comprise any useful solvent compatible with semiconductor manufacturing and with the organotin photoresist composition. Suitable solvents can include alcohols, ethers, esters, ketones, and combinations and mixtures thereof. Suitable organic solvents include, for example, alcohols or blends thereof. Generally, the solvents are at least 50 weight percent alcohols with any remaining organic solvent liquids being soluble in the alcohol, such as alkanes (such as pentane or hexane), aromatic hydrocarbons (such as toluene), ketones (such as methyl isobutyl ketone, cyclohexanone, butanone, and the like), ethers (such as propylene glycol methyl ether), esters (such as ethyl acetate, ethyl lactate, propylene glycol methyl ether acetate), or mixtures thereof. In some embodiments, the solvent is at least 90 weight percent alcohol, and the solvent can be effectively alcohol with just trace impurities of other compounds. In some embodiments, the solvent includes a primary alcohol. In some embodiments, the solvent includes two or more distinct alcohols. Suitable alcohols are generally monomeric alcohols with a melting point of no more than about 10° C., such as methanol, ethanol, propanol, butanol, pentanol, hexanol, heptanol, octanol, nonanol, decanol, branched versions thereof, and mixtures thereof. It has been found that controlling the water level can result in consistent and stable precursor solutions. In particular, the water level can be adjusted, generally by addition of small amounts of water to the solvent. to achieve the target water levels, generally no more than about 10,000 ppm by weight, and in additional embodiments from about 300 ppm by weight to about 2500 ppm by weight. A person of ordinary skill in the art will recognize that additional ranges within the explicit ranges above are contemplated and are within the present disclosure. The use of water content adjustment is discussed further in U.S. Pat. No. 11,300,876 (herein the '876 patent) to Jiang et al., entitled “Stable Solutions of Monoalkyl Tin Alkoxides and Their Hydrolysis and Condensation Products,” incorporated herein by reference.

While terminology referring to the additives may be suggestive, the specific terminology should not be interpreted as necessarily involving any specific mechanism of action, although certain classes of compositions may be acknowledged broadly in the arts. For example, while antioxidants are well known in the polymer arts, the acknowledgement as being antioxidants does not necessarily imply a particular mechanism of action other than they prevent polymer degradation generally associated with oxidative damage. The radical scavenging additives are based on their chemical formulation that suggests a desirable inhibition of changes to the radiation sensitive film associated conceptually with modification of the compositions resulting from radicals or other highly reactive species. Similarly, the various subgenus groupings for radical scavenging additives may similarly have suggestive names for the specific genus that again are not meant to imply the involvement of a specific mechanism or combinations thereof. Nevertheless, the additive compounds generally preferentially react with highly reactive species to form less reactive species.

While not wanting to be limited by theory, it is believed that the interaction of radicals with the organometallic photoresist material can lead to the undesirable cleavage of the tin-carbon bonds. The cleavage of the tin-carbon bond is generally undesirable in the absence of radiation exposure because tin-carbon bond cleavage is the main mechanism for generating contrast between irradiated and non-irradiated regions, which is driven by the radiation cleavage of the C—Sn bond. It is therefore desirable to include additives, such as radical scavenger compounds, that can inhibit the reactions of radicals or other highly reactive species with the photoresist material. Various compositional motifs provide the reaction with a radical or other reactive species to form a less reactive product, which may still be a radical, that can be quenched through benign mechanisms. These motifs include, for example, H-atom donating compounds, branched alkenes, conjugated unsaturated bonds, hindered phenols and derivatives thereof, aromatic diols, sterically hindered aminoxyl groups, hindered amines, organophosphine, phosphite esters, derivatives thereof and mixtures thereof, which are elaborated on in the following.

In some embodiments, the radical scavenger compounds can include, for example, compounds capable of donating an H atom, which may be effective to quench free radicals. Radical scavenger additives capable of donating an H atom (H-donors) to quench undesired radicals can be effective at neutralizing radicals that could otherwise undesirably react with the resist material. Such quenching can lead to less reactive products with less potential for driving undesired reactions within the resist. Specifically, desirable H-donor radical scavengers can react with and neutralize radicals such as R· or ROO· more readily than other R—H bonds (such as non-radical hydrocarbyl groups) to form stabilized radical species that generally do not react with non-radical species themselves. The stabilized radical species will then be stable enough to preferentially react with other radical species rather than propagating the radical reaction with non-radical species.

While not wanting to be limited by theory, the radical reaction schemes discussed above are shown below, where HT represents the H-donor radical scavenger. For example, when an H-donor radical scavenger reacts with an ROO· radical, it transfers a hydrogen atom to the radical, forming a hydroperoxide (ROOH) and a new, more stable radical derived from the H-donor. This new radical is typically less reactive due to various stabilizing factors such as resonance, hyperconjugation, or steric hindrance. As a result, it is less likely to continue the chain reaction by attacking other non-radical species in the photoresist composition. The stabilized radical species formed from the H-donor preferentially reacts with other radical species rather than non-radical components of the photoresist. This selectivity is useful for preserving the integrity of the organotin photoresist composition to quench the radical. By promoting radical-radical termination reactions, H-donors help to reduce the overall concentration of reactive radical species in the system and correspondingly reduce radical reactions with the resist composition.
ROO·+H-T→ROOH+T· (H transfer and radical abstraction),
T·+R·→termination,
T·+RH→little to no reaction.

H-donor radical scavenger additives can improve the stability of organotin photoresists by mitigating the effects of radical-induced degradation of the photoresist material. Thus, the incorporation of suitable H-donors into organotin photoresist formulations can significantly enhance the stability and performance of these materials in the presence of radical-generating contaminants. By effectively scavenging ROO· and other reactive radicals, H-donors help maintain the desired chemical and physical properties of the photoresist throughout the lithographic process, including precursor solution storage, ultimately contributing to improved stability, patterning fidelity, and reduced defectivity in semiconductor device fabrication. The results in the following Examples confirm that suitable radical scavenging additives can improve stability of organotin resists during storage.

Suitable H-donors for use in organotin photoresist compositions include compounds such as phenolic compounds and hindered amines. These molecules can be characterized by the presence of a substituted aromatic ring which can improve their ability to form relatively stable radicals after hydrogen atom transfer. While not wanting to be limited by theory, it is believed that the efficacy of H-donor radical scavengers can be improved by substitution of the aromatic ring with appropriate electron donating groups and/or by substitution of the aromatic ring with resonance-enhancing groups. Substitution of the aromatic ring with electron donating groups can lead to a lower the bond dissociation energy (BDE) of hydroxyl protons, i.e., the O—H bond, which can promote the H-transfer and radical abstraction processes to form a stabilized —O· radical. Substitution of the H-donor compound's aromatic ring with resonance-enhancing groups can stabilize the radical product by improving the delocalization of electron density through more resonance structures, and thereby reducing the O—H bond dissociation energy (BDE).

In some embodiments, the H-donor radical scavenger is a hindered phenol compound having an aromatic ring substituted with an electron-donating group. In some embodiments, the H-donor radical scavenger is a hindered phenol compound having an aromatic ring substituted with a resonance-enhancing group. In some embodiments, the hindered phenol compound can be represented by the structure

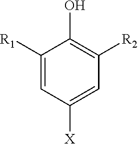

where R₁ and R₂ are independently alkyl groups having 1 to 4 carbon atoms and X is selected from —OH, —NH₂, —NMe₂, —OMe, —CH₂Sn(CH₃)₃. The electron-donating effects of the X group substituted on the aromatic ring can improve the lability of the hydroxyl proton, and thereby improving the H-donating ability of the compound. In some embodiments, X is an alkyl group having 1 to 4 carbon atoms. When X is an alkyl group, a resonance-enhancing effect is achieved by the formation of additional resonance structures which can stabilize the —O· radical and reduce the O—H BDE. When the radical scavenger compound has the structure above, the associated radical product comprises an —O· radical that is sterically hindered by the R¹ and R₂ groups at the ortho positions, which can stabilize the radical product and therefore promote the radical scavenging property of the H-donor radical scavenger. In some embodiments, the H-donor radical scavenger is butylated hydroxytoluene (BHT) or butylated hydroxyaniline (BHA).

In some embodiments, the H-donor radical scavenger is an aromatic diol having an OH at the ortho, meta, and/or para positions and represented by one of the structures

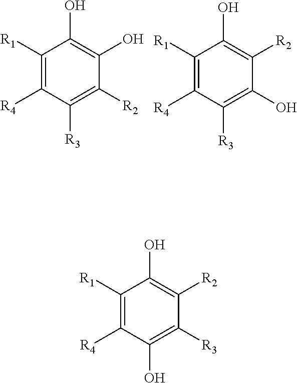

Where R₁, R₂, R₃, and R₄ are independently H, an alkyl group having 1 to 4 carbon atoms, or a halogen atom (e.g., F, Cl, I). In some embodiments, R¹ is OH and R₂, R₃, and R₄ are independently H or alkyl groups having 1 to 4 carbon atoms. In some embodiments R₁ and R₃ are H and R₂ and R₄ are tert-butyl. In some embodiments, at least one of R₁, R₂, R₃, and R₄ is tert-butyl. In some embodiments, the aromatic diol is represented by one of the structures

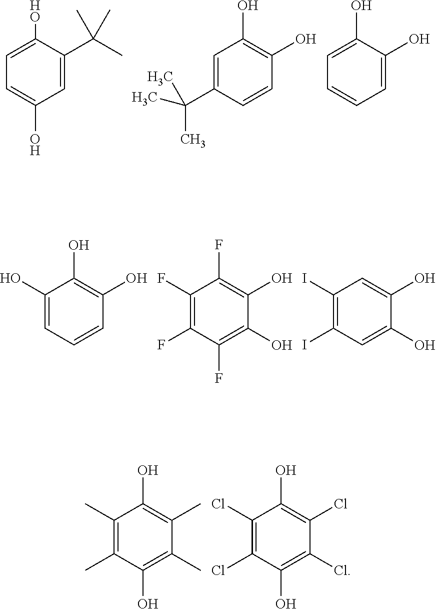

In some embodiments, the H-donor radical scavenger is an alkoxyphenol or derivative represented by the structure

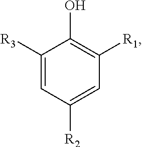

where R₁, R₂, and R₃ are independently H or —OR′ where R′ is an alkyl group having from 1 to 4 carbons, and where at least one of R₁, R₂, or R₃ is —OR′. In some embodiments, the radical scavenger is guaiacol where R₁ is —OMe and R₂ and R₃ are both H. Alkoxy groups, such as methoxy groups, are considered electron donating groups and when at least one of R₁, R₂, or R₃ is an alkoxy group (e.g., —OMe), the O—H BDE can be lowered, and the radical product can be stabilized. In the context of the above embodiments, the additive can comprise an aromatic ring substituted with an ether group (—OR′) having from 1 to 4 carbons and an alcohol group (—OH). Some non-limiting examples of suitable alkoxyphenols and derivatives include compounds having the structures

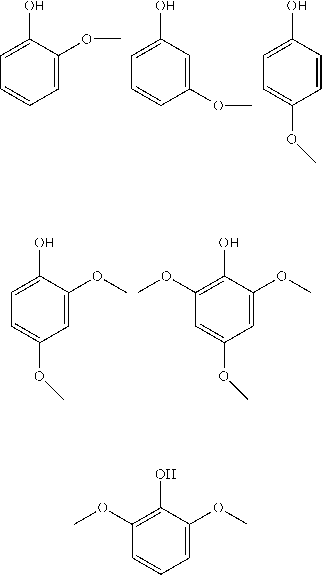

In some embodiments, the H-donor radical scavenger is a hindered aromatic amine compound represented by the structure

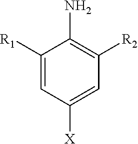

where R₁ and R₂ are independently alkyl groups having 1 to 4 carbon atoms and X is H or an alkyl group having 1 to 4 carbon atoms. In some embodiments, the hindered aromatic amine is 2,4,6-tri-tert-butyl aniline where R₁, R₂, and X are tert-butyl. The aromatic ring can improve the stability of the radical product through resonance stabilization, and further substitution of the ring with resonance-enhancing alkyl groups such as tert-butyl can further improve the stability of the radical product.

The amine functional group can provide for an electron donating effect to the aromatic ring, which can improve the stability of the radical product. Additionally, the substituted aromatic ring structure provides for resonance stabilization, which improves the stability of the radical product formed. This resonance stabilization allows the unpaired electron to be delocalized across the aromatic system, reducing its reactivity and hindering further undesired reactions within the photoresist material.

In particular, bulky alkyl substituents such as tert-butyl can be particularly effective at improving the radical scavenging ability of the hindered aromatic amine. Some advantages of aromatic amines substituted with alkyl groups include steric hindrance of reactions of the radical product with other molecules and hyperconjugation which can further delocalize the unpaired electron to contribute to the overall stability of the radical product. By forming more stable radical products that are less likely to propagate chain reactions while also maintaining their ability to terminate other radicals, hindered aromatic amine compounds such as 2,4,6-tri-tert-butyl aniline and similar compounds with substituted aromatic rings can significantly improve the stability and performance of organotin photoresist compositions.

In some embodiments, the additive is a hindered amine compound. The hindered amine compound can react with oxygen or reactive oxygen species to form a stable and sterically hindered aminoxyl (—NO·) radical which can preferentially react with other radical species instead of the non-radical components of the photoresist. A useful form of hindered amines is found in the following formula.

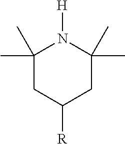

where R is H, OH, or a linear, branched, cyclic, or aromatic alkyl group having from 1 to 10 carbon atoms. In some embodiments R is H and reacts with oxygen and/or a reactive oxygen species (ROS) to form (2,2,6,6-Tetramethylpiperidin-1-yl)oxyl (TEMPO). In some embodiments, R is OH and reacts with oxygen and/or an ROS to form (4-Hydroxy-2,2,6,6-Tetramethylpiperidin-1-yl)oxyl (TEMPOL). In some embodiments, the radical scavenger additive is TEMPO.

The use of hindered amine compounds as radical scavengers in organotin photoresist compositions is advantageous for mitigating the effects of radical-induced degradation. These compounds are particularly effective due to their ability to form stable aminoxyl radicals when exposed to oxygen radicals or reactive oxygen species. The resulting aminoxyl radicals, such as (2,2,6,6-Tetramethylpiperidin-1-yl)oxyl (TEMPO), possess exceptional stability. In TEMPO, the N—H group in the above formula is replaces with N—O·. Such radicals can be remarkably stable due to the presence of bulky substituents around the nitrogen atom, which provide steric protection and can prevent unwanted side reactions. The stability of the aminoxyl radical is further enhanced by the delocalization of the unpaired electron between the nitrogen and oxygen atoms. Once formed, these aminoxyl radicals can serve as efficient radical traps. They can preferentially react with other radical species present in the photoresist environment or matrix, effectively neutralizing these potentially harmful radicals before they can interact with and degrade the organotin components of the photoresist. In some embodiments, the radical scavenger additive is 2-Phenyl-4,4,5,5-tetramethylimidazoline-1-oxyl 3-oxide (PTIO).

In some embodiments, the radical scavenging additives can include compounds having a branched alkene structure, such as represented by the structure

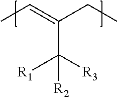

where R₁, R₂, and R₃ are independently H or CH₃ and the brackets indicate optional additional bonding. In some embodiments, the additive can include compounds having a secondary or tertiary carbon adjacent to a branched alkene structure such as in the structure above wherein at least R₁ and R₂ are CH₃. Such branched alkene structures can enhance the stability of the additive after reaction with the radical species, thereby increasing the effectiveness of the additive. In some embodiments, the additive can include a compound having both a branched alkene bond structure and a ketone and/or an alcohol functional group. Additives having a branched alkene structure can include squalene and α- and β-ocimene. Additives having a branched alkene bond structure and a ketone and/or an alcohol functional group include alpha-Ionone and trans,trans-Farnesol.

In some embodiments, the radical scavenging additive can include, for example, compounds having conjugated double bonds. While not wanting to be limited by theory, it is believed that the π-electrons in conjugated systems can act as electron donors or acceptors and can effectively scavenge free radicals like those formed from interaction of the photoresist with radical-generating contaminants. The radical scavenging action provided by conjugated double bonds systems can neutralize radicals generated by radical-generating contaminants species and can prevent undesired chemical changes in the resist. Tin ligands with conjugated double bonds have been made and used for patterning, as described in copending U.S. provisional patent application 63/557,166 to Jilek et al., entitled “Organometallic Compositions With Diene Ligands And Patterning,” incorporated herein by reference.

In some embodiments, the radical scavenging additive can include an organophosphine compound having the formula PR₃, where R is a linear, branched, cyclic, or aromatic alkyl group having from 1 to 10 carbon atoms. In some embodiments, the additive can include triphenyl phosphine, which is known to neutralize peroxides.

In some embodiments, the radical scavenging additive can include a phosphite ester compound having the formula P(OR)₃, where R is a linear, branched, cyclic, or aromatic alkyl group having from 1 to 31 carbon atoms. Phosphite esters are used in polymers as antioxidants since they preferentially undergo stabile oxidation. Some suitable examples of phosphite ester compounds include trimethyl phosphite, triethyl phosphite, triphenyl phosphite, tris(4-methylphenyl)phosphite, tris(4-tert-butylphenyl)phosphite, tris(2,4,6-methylphenyl)phosphite, and tris(2,4-di-tert-butylphenyl)phosphite.

As can be seen above, a wide range of compounds are known that can provide suitable radical scavenging or associated function as an additive. For stabilization of precursor solution, selection can be guided in part by solubility in the selected solvent. Over the range of compounds, the properties span a large range. Gas phase or low boiling point additives can be suitable for stabilizing the solutions in closed containers to inhibit evaporation and are naturally removed by evaporation during coating of the composition to make the film. Other higher boiling point additives can be maintained in the film from the precursor solutions or deposited by vapor deposition. Generally, film processing involves heating steps with a general trend toward higher temperature processing. An additive can be selected to persist until removed by heating at a particular stage. Heating temperatures are generally higher after irradiation, and the removal of a radical scavenging additive can take place at these higher temperature bake steps after irradiation. Thus, the radiation scavenging additive can have a boiling point or decomposition temperature greater than 100° C., in further embodiments from about 120° C. to about 400° C., in additional embodiments from about 140° C. to about 350° C., in further embodiments form about 150° C. to about 300° C., and in other embodiments greater than about 250° C. to persist to a hard bake or following a hard bake. For radical scavenging additives to be removed prior to irradiation, it can be desirable to have a boiling point of no more than about 200° C. and in further embodiments form about 55° C. to about 160° C. While some additives can be maintained after a final hard bake step, generally it is desirable to have the additive removed by a final hard bake if not sooner. A person of ordinary skill in the art will recognize that additional ranges of temperature within the explicit ranges above are contemplated and are within the present disclosure.

### Radiation Sensitive Films and Processes for the Films

As noted above, the organotin MO precursors can be deposited from solution or vapor. For solution deposition, the photoresist precursor solutions with organotin compositions and radical scavenging additives can be used to form radiation-patternable organotin oxo hydroxo materials incorporating the radical scavenging additives or with the additives removed during processing by evaporation, and such coatings can be formed using any suitable method known in the art. Spin coating can be particularly desirable for forming coatings using the photoresist precursor solutions with radical sequestering additives. In a typical spin coating process, a volume of a photoresist solution is introduced onto the surface of a substrate, and the substrate is rotated at high speeds to drive rapid evaporation and hydrolysis processes to enable the formation of a radiation patternable coating. In some embodiments, the substrate can be spun at rates (i.e., spin speeds) from about 500 rpm to about 10,000 rpm, in further embodiments from about 1000 rpm to about 7500 rpm, and in additional embodiments from about 2000 rpm to about 6000 rpm. The spin speed can be adjusted to obtain a desired coating thickness. The spin coating can be performed from about 5 seconds to about 5 minutes and in further embodiments from about 15 seconds to about 2 minutes. An initial low speed spin, e.g., at 50 rpm to 250 rpm, can be used to perform an initial bulk spreading of the composition across the substrate. A back side rinse, edge bead removal step, or the like can be performed with water or other suitable solvent to remove any edge bead. A person or ordinary skill in the art will recognize that additional ranges of spin coating parameters within the explicit ranges above are contemplated and are within the present disclosure.

A substrate generally presents a surface onto which the coating material can be deposited, and the substrate may comprise a plurality of layers in which the surface relates to an upper most layer. The substrate surface can be treated to prepare the surface for adhesion of the coating material. Prior to preparation of the surface, the surface can be cleaned and/or smoothed as appropriate. Suitable substrate surfaces can comprise any reasonable material. Some substrates of interest include, for example, silicon wafers, silica substrates, other inorganic materials, polymer substrates, such as organic polymers, composites thereof and combinations thereof across a surface and/or in layers of the substrate. In some embodiments, the substrate can comprise a patterned structure such as described by Stowers et al. in U.S. Pat. No. 10,649,328, entitled “Pre-Patterned Lithography Templates, Process Based on Radiation Patterning Using The Templates And Processes To Form The Templates”, incorporated herein by reference.

The thickness of the coating generally can be a function of the precursor solution concentration, viscosity and the spin speed for spin coating. For other coating processes, the thickness can generally also be adjusted through the selection of the coating parameters. As noted above, many precursors can be deposited by vapor deposition, and flow rates, times, pressures and other parameters can be adjusted to yield a desired coating.

Owing generally to their high vapor pressures, the organotin compositions described herein can be useful as precursors for forming coatings via vapor deposition. Vapor deposition methods generally include chemical vapor deposition (CVD), physical vapor deposition (PVD), atomic layer deposition (ALD), and modifications thereof. In a typical vapor deposition process, the organotin composition can be reacted with small molecule gas-phase reagents such as H₂O, O₂, H₂O₂, O₃, CH₃OH, HCOOH, CH₃COOH, and the like, which serve as 0 and H sources for production of radiation sensitive organotin oxide and oxide hydroxide coatings and the like. Vapor deposition of radiation patternable organotin coatings has been described in the '618 patent cited above, as well as by Wu et. al in PCT Application #PCT/US2019/031618 entitled “Methods for Making EUV Patternable Hard Masks”, incorporated herein by reference. Production of radiation sensitive organotin coatings can generally be achieved by reacting the volatile organotin precursor RSnL₃ with a small gas-phase molecule. The reactions can include hydrolysis/condensation of the organotin precursor to hydrolyze the hydrolysable ligands while leaving the Sn—C bonds substantially intact.

For radical scavenging additives, if they have sufficient vapor pressure, the additives can be introduced into the coating chamber at a desired partial pressure to deposit a selected amount of additive into the coating. The radical scavenging additive can be codeposited or sequentially deposited with the precursor. So for vapor deposition of the precursor, the additive can be simultaneously deposited in a selected concentration. In ALD, the radical scavenging additive deposition can be performed with a layer, between layers or any suitable combination. For solution deposited precursors, the radical scavenging additive can be deposited by vapor deposition onto (sequentially after) or under (sequentially before) the precursor, as well as a combination thereof (before, during and/or after). A post application heating can provide an anneal of the material to provide more uniformity for further processing.

In some embodiments, it can be desirable to use a thin coating to facilitate formation of small and highly resolved features in the subsequent patterning process. For example, the coating materials after drying can have an average thickness of no more than about 250 nanometers (nm), in additional embodiments from about 1 nm to about 50 nm, in other embodiments from about 2 nm to about 40 nm and in further embodiments from about 3 nm to about 25 nm. A person of ordinary skill in the art will recognize that additional ranges of thicknesses within the explicit ranges above are contemplated and are within the present disclosure. The thickness can be evaluated using non-contact methods of x-ray reflectivity and/or ellipsometry based on the optical properties of the film. In general, the coatings are relatively uniform to facilitate processing. In some embodiments, such as high uniformity coatings on reasonably sized substrates, the evaluation of coating uniformity or flatness may be evaluated with, for example, a 1 centimeter edge exclusion, i.e., the coating uniformity is not evaluated for portions of the coating within 1 centimeter of the edge, although other suitable edge exclusions can be selected.

While heating may not be needed for successful application of the deposition process, it can be desirable to heat the coated substrate to densify the coating material, to speed the processing, to increase the reproducibility of the process, and/or to facilitate vaporization of the hydrolysis by-products, such as alcohols and/or amines. A post application bake may also be used to remove radical scavenging additives or a portion thereof used to stabilize precursors solutions, prior to irradiation In embodiments in which heating of the coated substrate is performed prior to irradiation, the coated substrate can be heated to temperatures from about 45° C. to about 250° C., in further embodiments from about 55° C. to about 225° C., and in additional embodiments from about 65° C. to about 200° C. The heating can generally be performed for at least about 0.1 minute, in further embodiments for about 0.5 minutes to about 30 minutes, and in additional embodiments from about 0.75 minutes to about 10 minutes. The heating may be performed in air, vacuum, or an inert gas ambient, such as Ar or N₂. A person of ordinary skill in the art will recognize that additional ranges of heating temperatures and times within the explicit ranges above are contemplated and are within the present disclosure.

Generally, photoresist coatings can be patterned using radiation. Suitable radiation sources include extreme ultraviolet (EUV), ultraviolet (UV), or electron beam (EB) radiation. For fabrication of semiconductor devices, EUV radiation can be desirable due to its higher resolution compared to UV radiation, and its higher throughput compared to electron beam (EB)-based processing. While the short wavelength of EUV radiation, is effective for fine patterning, the high energy of the photons has the potential of generating highly reactive species, such as radicals, the control of which is the present focus. Radiation can generally be directed to the substrate material through a mask or a radiation beam can be controllably scanned across the substrate to form a latent image within the resist coating. For EUV “masking,” the patterns can be formed using mirrors to reflect and direct light from a plasma source.

In general, while suitable radiation sources are those that generally provide for wavelengths that effectively absorb in the photoresist, typical radiation sources generally correspond to commercial lithography applications. For example, wavelengths most relevant to lithography include commercial EUV exposure tools (such as those fabricated by ASML) which operate at a wavelength of 13.5 nm and commercial UV exposure tools which generally operate at a wavelength of 193 nm for ArF excimer laser sources or 248 nm for KrF excimer laser sources. A person of ordinary skill in the art will understand that other absorbative wavelengths are contemplated and within the scope of the disclosure. The international standard for optics and photonics is ISO 20473:2007(E), incorporated herein by reference. This standard has the broad range of UV wavelengths from 1 nm to 380 nm, with the EUV range from 1 nm to 100 nm.

The amount of electromagnetic radiation can be characterized by a fluence or dose which is obtained by the integrated radiative flux over the exposure time. For embodiments in which EUV radiation is used, suitable radiation doses can be from about 1 mJ/cm² to about 150 mJ/cm², in further embodiments from about 2 mJ/cm² to about 100 mJ/cm² and in further embodiments from about 3 mJ/cm² to about 50 mJ/cm². A person of ordinary skill in the art will recognize that additional ranges of radiation fluences within the explicit ranges above are contemplated and are within the present disclosure.

Following exposure to radiation and the formation of a latent image, a subsequent postexposure bake (PEB) is typically performed. In some embodiments, the PEB can be performed at temperatures from about 45° C. to about 250° C., in additional embodiments from about 50° C. to about 220° C., in some embodiments from about 80° C. to about 200° C. and in further embodiments from about 90° C. to about 185° C. The post exposure heating can generally be performed for at least about 0.1 minute, in further embodiments from about 0.5 minutes to about 30 minutes and in additional embodiments from about 0.75 minutes to about 10 minutes. The heating may be performed in air, vacuum, or an inert gas ambient, such as Ar or N₂, and heating steps can be broken into separate heating steps with different gaseous environments. In some embodiments, multiple PEB steps can be performed where the coating material is heated at different temperatures and/or in different ambient environments. For selected radical scavenging additives, these compounds would be expected to evaporate from the films at some PEB temperatures. A person of ordinary skill in the art will recognize that additional ranges of PEB temperatures and times within the explicit ranges above are contemplated and are within the present disclosure. The PEB can be designed to further consolidate the exposed regions without decomposing the un-exposed regions into a metal oxide.

Following performing a PEB, development of the image involves the contact of the patterned coating material including the latent image to a developer composition to remove either the un-irradiated coating material to form the negative image or the irradiated coating to form the positive image. Irradiated regions of organotin oxide hydroxide coatings are generally hydrophilic and are thus soluble in aqueous bases and insoluble in organic solvents; conversely, non-irradiated regions are generally hydrophobic and are thus soluble in organic solvents and insoluble in aqueous bases. For negative tone imaging, the developer can be an organic solvent, such as the solvents used to form the precursor solutions.

Specifically, for positive tone imaging, suitable developers generally can be aqueous bases. In some embodiments, aqueous bases can be used to obtain sharper images. To reduce contamination from the developer, it can be desirable to use a developer that does not have metal atoms. Thus, quaternary ammonium hydroxide compositions, such as tetraethylammonium hydroxide, tetrapropylammonium hydroxide, tetrabutylammonium hydroxide or combinations thereof, are desirable positive tone developers. In general, the quaternary ammonium hydroxides of particular interest can be represented with the formula R₄NOH, where R=a methyl group, an ethyl group, a propyl group, a butyl group, or combinations thereof. The coating materials described herein generally can be developed with the same developer commonly used presently for polymer resists, specifically tetramethyl ammonium hydroxide (TMAH). Commercial TMAH is available at 2.38 weight percent. Furthermore, mixed quaternary tetraalkyl-ammonium hydroxides can be used. In general, the developer can comprise from about 0.5 to about 30 weight percent, in further embodiments from about 1 to about 25 weight percent and in other embodiments from about 1.25 to about 20 weight percent tetra-alkylammonium hydroxide or similar quaternary ammonium hydroxides. A person of ordinary skill in the art will recognize that additional ranges of developer concentrations within the explicit ranges above are contemplated and are within the present disclosure. For a positive tone developer, it can be desirable to dissolve material densified from a relatively high radiation dose, assuming that the non-irradiated material is not significantly removed. This opens up the process window further.

For the negative tone imaging, the developer can be an organic solvent, such as the solvents used to form the precursor solutions. For negative tone development, developer selection can be influenced by solubility parameters with respect to the coating material, both irradiated and non-irradiated, as well as developer volatility, flammability, toxicity, viscosity and potential chemical interactions with other process material. In particular, suitable developers include, for example, aromatic compounds (e.g., benzene, xylenes, toluene), esters (e.g., propylene glycol monomethyl ester acetate (PGMEA), ethyl acetate, ethyl lactate, n-butyl acetate, butyrolactone), alcohols (e.g., 4-methyl-2-pentanol, 1-butanol, isopropanol, 1-propanol, methanol), ketones (e.g., methyl ethyl ketone, acetone, cyclohexanone, 2-heptanone, 2-octanone), ethers (e.g., tetrahydrofuran, dioxane, anisole) and the like. Improved developer compositions have been described in published U.S. Patent Application No.: 2020/0326627 to Jiang et al., entitled “Organometallic Photoresist Developer Compositions and Processing Methods,” incorporated herein by reference. Improved developer solutions generally comprise a reference organic solvent composition and an additive composition having a higher polarity and/or hydrogen-bonding character than the reference solvent composition. In one example, an improved developer composition can comprise PGMEA and acetic acid. The development can be performed for about 5 seconds to about 30 minutes, in further embodiments from about 8 seconds to about 15 minutes and in addition embodiments from about 10 seconds to about 10 minutes. A person of ordinary skill in the art will recognize that additional ranges within the explicit ranges above are contemplated and are within the present disclosure.

Some useful developer compositions for negative tone development of these organotin oxide photoresists have been described in published U.S. Patent Application No. 2020/0326627 to Jiang et al., entitled “Organometallic Photoresist Developer Compositions and Processing Methods”, incorporated herein by reference. In particular, developers can have differing amounts of more polar or less polar components, which can be specified more specifically with solubility parameters. In some embodiments, the solvent blend can comprise at least two solvents with at least 55 volume % of one or more solvents each independently having a sum of Hansen solubility parameter δH+δP of no more than about 16 (J/cm³)^{1/2}, and with from about 0.25 volume % to about 45 volume % of one or more solvents each independently having a sum of Hansen solubility parameter δH+δP of at least about 16 (J/cm³)^{1/2}. For double bake-double develop processing, a second development can involve a developed for a negative tone pattern with a developer that can comprise all or a larger percentage of a solvent having a sum of Hansen solubility parameter δH+δP of at least about 16 (J/cm³)^{1/2}, or a positive tone developer can be used in the second step of a negative tone patterning.

It has also been discovered that solventless development, also referred to as dry development, can be employed with organotin materials. Dry development can include, for example, selective removal of the irradiated or non-irradiated regions of the photoresist by exposing the material to an appropriate plasma or appropriate flowing gas. Dry development of organotin resists has been described in PCT Publication No. 2020/132281A1 by Volosskiy et al., entitled “Dry Development of Resists”, and in published U.S. Patent Application No. 2023/0100995 to Cardineau et al., entitled “High Resolution Latent Image Processing and Thermal Development”, both of which are incorporated herein by reference. In such dry development processes, development can be achieved by exposing the irradiated substrate to a plasma or a thermal process while flowing a gas comprising a small molecule reactant that facilitates removal of irradiated or non-irradiated regions. Dry development can also be based on halide chemistries, as described by Tan et. al in PCT Pat App. No: PCT/US2020/039615 entitled “Photoresist Development With Halide Chemistries”, incorporated herein by reference. For organotin photoresist coatings, dry development can be conducted through the use of halogen-containing plasmas and gases, for example HBr and BCl₃. In some cases, dry development may offer advantages over wet development such as reduced pattern collapse, deceased scum, and fine control over developer compositions, i.e. the plasma and/or etch gases. See also, published U.S. patent application 2023/0408916 to De Schepper et al., entitled “Gas-Based Development of Organometallic Resist in an Oxidizing Halogen-Donating Environment,” incorporated herein by reference. Following development, a rinse step can be conducted if desired to further remove undesired material from the pattern, and such methods have been described in published U.S. Patent Application No. 2020/0124970 to Kocsis et al., entitled “Patterned Organometallic Photoresists and Methods of Patterning,” incorporated herein by reference.

After completion of the development step including any optional rinses, the coating materials can be heat treated to further condense the material and to further dehydrate, densify, or remove residual developer from the material. This heat treatment can be particularly desirable for embodiments in which the oxide coating material is incorporated into the ultimate device, although it may be desirable to perform the heat treatment for some embodiments in which the coating material is used as a resist and ultimately removed if the stabilization of the coating material is desirable to facilitate further patterning. In particular, the bake of the patterned coating material can be performed under conditions in which the patterned coating material exhibits desired levels of etch selectivity. In some embodiments, the patterned coating material can be heated at a temperature from about 100° C. to about 600° C., in further embodiments from about 175° C. to about 500° C. and in additional embodiments from about 200° C. to about 400° C. The heating can be performed for at least about 1 minute, in other embodiment for about 2 minutes to about 1 hour, in further embodiments from about 2.5 minutes to about 25 minutes. The heating may be performed in air, vacuum, or an inert gas ambient, such as Ar or N₂. A person of ordinary skill in the art will recognize that additional ranges of temperatures and time for the heat treatment within the explicit ranges above are contemplated and are within the present disclosure. Likewise, nonthermal treatments, including blanket UV exposure, or exposure to an oxidizing plasma such as 02 may also be employed for similar purposes.

EUV lithography is generally used for very small resolution features to take advantage of the small wavelength of the light. Patterning for device formation generally is leveraged from an initial pattern that can be effectively formed on the substrate. The patterns can generally depend on the ultimate device's function (for example, logic or memory) and can comprise a variety of features, for example, stripes, posts, holes, geometric shapes, and other features. Generally, test patterns used to analyze photoresist performance can include patterning of line-space patterns, arrays of pillars, or arrays of holes.

In some embodiments involving stripes, such as line-space patterns, and patterned with radiation (UV, EUV or e-beam), adjacent linear segments of neighboring structures can have an average pitch (half-pitch) of no more than about 250 nm (125 nm half-pitch), in some embodiments no more than about 100 nm (50 nm half-pitch) and in further embodiments no more than about 60 nm (30 nm half-pitch). Pitch can be evaluated by design and confirmed with scanning electron microscopy (SEM), such as with a top-down image. Patterns can be evaluated, for example, with a Hitachi CG5000 CD-SEM instrument, or the like. As used herein, pitch refers to the spatial period, or the center-to-center distances of repeating structural elements, and as generally used in the art a half-pitch is a half of the pitch. Feature dimensions of a pattern can also be described with respect to the average width of the feature, which is generally evaluated away from corners or the like. The line widths are also referred to as a critical dimension (CD), and the critical dimension can be used to evaluate available feature sizes for a particular dose. For holes on a grid, a corresponding critical dimension is the hole diameter. Also, features can refer to gaps between material elements and/or to material elements. In some embodiments, average widths can be no more than about 25 nm, in further embodiments no more than about 20 nm, and in additional embodiments no more than about 15 nm. A person of ordinary skill in the art will recognize that additional ranges of pitch and average widths within the explicit ranges above are contemplated and are within the present disclosure. Based on these and related processes, the patterning can be adapted to the formation of various devices such as electronic integrated circuits, generally through the repeated patterning process to form appropriately layered structures, such as transistors or other components.

Wafer throughput is a substantially limiting factor for implementation of EUV lithography in high-volume semiconductor manufacturing, and is directly related to the dose required to pattern a given feature. However, while chemical strategies exist to reduce imaging dose, a negative correlation between the imaging dose required to print a target feature, and feature size uniformity (such as LWR) is commonly observed for EUV photoresists at feature sizes and pitches <50 nm, thereby limiting final device operability and wafer yields. Photoresist sensitivity can be expressed in terms of the dose-to-gel value, and imaging dose requirements can be evaluated by forming an array of exposed pads in which the exposure time is stepped from pad to pad to change the dosing of the exposure, which can be referred to as dose meander exposure. The film can then be developed, and the thickness of the remaining resist can be evaluated for all of the pads, for example, using spectroscopic ellipsometry. The measured thicknesses can be normalized to the maximum measured resist thickness and plotted versus the logarithm of exposure dose to form characteristic contrast curves. The maximum slope of the normalized thickness vs log dose curve is defined as the photoresist contrast (γ), and the dose value at which a tangent line drawn through this point equals 1 is defined as the photoresist dose-to-gel, (D_{g}). Another parameter that can be evaluated from the dose meander exposure with patterned exposure rather than a uniform exposure is the dose to size. After development, the feature spacing, or critical dimension, can be measured for each dose, and the dose is determined that results in the target feature size, and the examples include measurements of dose-to-size evaluations and variations depending on the additive. Common parameters used for photoresist characterization may be approximated following Mack, C. Fundamental Principles of Optical Lithography, John Wiley & Sons, Chichester, U.K; pp 271-272, 2007. EUV image analysis is described further in P. De Bisschop and E. Hendrickx “Stochastic effects in EUV lithography”, Proc. SPIE 10583, Extreme Ultraviolet (EUV) Lithography IX, 105831K (19 Mar. 2018); https://doi.org/10.1117/12.2300541, incorporated herein by reference.

### EXAMPLES

### Example 1: Preparation of Photoresist Precursor Solutions and Deposition of Films

This example presents the general preparation of the additive-containing photoresist precursor solutions and deposited photoresist films.

A series of photoresist precursor solutions were prepared by first dissolving and mixing appropriate masses of tert-butyltin tris(n-pentoxide) precursor and iso-propyltin tris (sec-butoxide) precursor in a 4-methyl-2-pentanol solvent to achieve a molar ratio of 80% tBuSn:20% i-PrSn at a total concentration of 0.05 M Sn for all photoresist solutions. Prior to addition of the organotin precursors, the solvent was initially mixed with a water source to normalize the solvent to a water content of 300 ppm using the methods described in the '876 patent. Appropriate masses of the additives described in Table 1 were then added and thoroughly mixed to form photoresist precursor solutions having the targeted molar ratios of additive to Sn concentration according to Table 2. Photoresist precursor solution control or comparative sample “CE” was prepared without any additive.

TABLE 1

| | | Approximate |
|---|---|---|
| Additive | Compound | Boiling Point, ° C. |
| S1 | butylated hydroxy toluene, BHT | 265 |
| S2 | (2,2,6,6-Tetramethylpiperidin-1-yl)oxyl, TEMPO | 193 |
| S3 | 4-Hydroxy-2,2,6,6-tetramethylpiperidine-1-oxyl, | 302 |
| | TEMPOL | |
| S4 | 2-tert-butyl-1,4-benzoquinone, TBQ | 273 |

TABLE 2

| Photoresist Precursor | | Molar Ratio |
|---|---|---|
| Solution Sample | Additive | (Additive:Sn) |
| A1 | S1 | 0.05:1 |
| A2 | S1 | 0.5:1 |
| B1 | S2 | 0.05:1 |
| B2 | S2 | 0.1:1 |
| B3 | S2 | 0.2:1 |
| C1 | S3 | 0.05:1 |
| C2 | S3 | 0.1:1 |
| C3 | S3 | 0.2:1 |
| D1 | S4 | 0.1:1 |
| D2 | S4 | 0.2:1 |
| D3 | S4 | 0.3:1 |
| CE | n/a | n/a |

A series of photoresist films were then prepared by spin-coating each photoresist precursor solution sample, according to Table 3, onto a 300 mm Si wafer pre-coated with an approximately 10 nm spin-on-glass (SOG) film. Following deposition, the wafers were subjected to a post-apply bake (PAB) of 100° C. for 60 seconds. The films were then analyzed via ellipsometry to determine the average film thicknesses and the results are presented in Table 3.

TABLE 3

| | Photoresist | Average | |
|---|---|---|---|
| | Precursor | Film | Change in |
| Photoresist | Solution | Thickness | Average Film |
| Film Sample | Sample | (nm) | Thickness |
| FA1 | A1 | 23.7 | 2.2% |
| FA2 | A2 | 24.3 | 4.7% |
| FB1 | B1 | 23.3 | 0.4% |
| FB2 | B2 | 23.3 | 0.4% |
| FB3 | B3 | 23.6 | 1.7% |
| FC1 | C1 | 23.6 | 1.7% |
| FC2 | C2 | 23.6 | 1.7% |
| FC3 | C3 | 23.8 | 2.6% |
| FD1 | D1 | 23.6 | 1.7% |
| FD2 | D2 | 23.8 | 2.6% |
| FD3 | D3 | 24.3 | 4.7% |
| FCE | CE | 23.2 | n/a |

The results show that the additives modestly increase the resulting film thickness relative to the resist film without any additive (FCE). The percent increase in average film thickness as compared to the films with additives (FCE) ranged from 0.4% to 4.7%. The film prepared with BHT additive at a molar ratio of 0.05:1 (FA1) showed a higher increase in average thickness (2.2% increase) than the films prepared with TEMPO (FB1, 0.4% increase) or TEMPOL (FC1, 1.7% increase) at the same molar ratio. Films prepared with TEMPO additive at molar ratios of 0.1 and 0.2 (FB2 and FB3, respectively) showed lower increases in average thickness (0.4% and 1.7%, respectively) than the films prepared with TEMPOL (FC2, 1.7% increase and FC3, 2.6% increase) and TBQ (FD1, 1.7% increase and FD2, 2.6% increase) at the same molar ratios.

This example demonstrates the preparation of the additive-containing photoresist precursor solutions and deposited photoresist films, which showed only small changes in coating thickness as compared to non-additive, comparative films.

### Example 2: Patterning and Patterning Stability Improvements

This example shows the effect of various radical scavenging additives on the dose-to-size (DtS)/CD stability of photoresist films subject to a post-exposure delay.

A series of photoresist film samples were prepared from photoresist precursor solutions, as described in Example 1. Six photoresist film samples were prepared from each photoresist precursor solution, to provide three sets of film samples (two film samples per set) for each coating composition, as shown in Table 4. Each film sample was analyzed in duplicate. Line-space patterns having a target critical dimension (CD) of 16 nm on a 32 nm pitch (16p32) were produced for each sample by exposing the samples to EUV radiation using an ASML NXE3400B exposure tool to create an array of patterns within fields on the wafer, wherein each field corresponds to the mask pattern printed at a specific dose. As is customary in the art, this type of exposure is referred to as a dose meander exposure. By exposing the same 16p32 pattern at different doses across each photoresist film sample, the dose required for printing the desired 16p32 pattern for a given photoresist film sample (i.e., the dose-to-size for printing 16 nm lines on a 32 nm pitch) can be determined through inspection of each field after processing is complete. Following EUV exposure, one of each of the duplicate film samples from each set were immediately processed by performing a PEB at 180° C., 200° C. or 210° C., dynamic development with a solution of 5 wt % acetic acid in PGMEA for approximately 30 s seconds, and a hard bake at 250° C. for 60 seconds to form developed negative tone images. Each of the other duplicate photoresist film samples from each set were subjected to a 2-hour delay in air following exposure but prior to performing the same PEB, development, and hard bake steps.

After the hard bake, the film samples were inspected using a Hitachi CD-SEM to determine the dose required to image the 16p32 pattern. The dose required to image the target 16p32 line-space pattern can be referred to as dose-to-size (DtS). At dose values less than DtS, the lines within the 16p32 pattern are less than 16 nm. Conversely, at dose values greater than DtS, the lines within the 16p32 pattern are greater than 16 nm. The DtS values for both the film samples processed immediately (without a post-EUV delay) and the film samples processed with a two-hour post-EUV delay in air were collected, as shown in Table 4. Table 4 also records the calculated percent change in DtS for each sample due to the two-hour delay after EUV exposure. The DtS change shows the efficacy of the additives in mitigating changes in DtS due to processing delay in air.

TABLE 4

| Photoresist | | DtS without | DtS with two- | DtS |
|---|---|---|---|---|
| Precursor | | delay | hour delay in | Change, |
| Solution Sample | PEB, ° C. | (mJ/cm2) | air (mJ/cm2) | % |
| A1 | 180 | 89.3 | 85.5 | −4.45 |
| | 200 | 73.9 | 71.1 | −3.93 |
| | 210 | 63.3 | 61.5 | −2.90 |
| A2 | 180 | 118.5 | 111.2 | −6.60 |
| | 200 | 82.0 | 82.8 | 0.94 |
| | 210 | 68.6 | 69.3 | 0.96 |
| B1 | 180 | 84.8 | 80.0 | −5.97 |
| | 200 | 68.4 | 67.6 | −1.26 |
| | 210 | 59.5 | 58.8 | −1.19 |
| B2 | 180 | 84.7 | 82.1 | −3.15 |
| | 200 | 69.4 | 68.1 | −1.85 |
| | 210 | 61.1 | 59.3 | −2.97 |
| B3 | 180 | 88.8 | 85.0 | −4.41 |
| | 200 | 70.9 | 69.4 | −2.18 |
| | 210 | 62.1 | 59.1 | −5.08 |
| C1 | 180 | 88.9 | 85.2 | −4.34 |
| | 200 | 72.0 | 70.7 | −1.78 |
| | 210 | 63.0 | 60.3 | −4.42 |
| C2 | 180 | 104.1 | 94.4 | −10.30 |
| | 200 | 76.4 | 74.8 | −2.07 |
| | 210 | 65.8 | 62.4 | −5.48 |
| C3 | 200 | 86.9 | 84.2 | −3.23 |
| | 210 | 70.1 | 67.6 | −3.81 |
| D1 | 200 | 94.0 | 96.2 | 2.31 |
| | 210 | 78.2 | 78.0 | −0.31 |
| D2 | 200 | 111.5 | 113.2 | 1.55 |
| | 210 | 94.8 | 87.5 | −8.41 |
| D3 | 210 | 110.0 | 100.5 | −9.48 |
| CE | 180 | 79.3 | 67.0 | −18.25 |
| CE | 200 | 65.5 | 55.9 | −17.21 |
| CE | 210 | 56.0 | 47.3 | −18.33 |

A DtS change of 0 would indicate that the delay in air had no impact on the patterning dose needed to achieve 16p32 line-space patterns. For the baseline photoresists without any additive (prepared from photoresist solution sample CEL), the DtS change due to processing delay is −18.25%, −17.21%, and −18.33% at PEBs of 180° C., 200° C., and 210° C., respectively, indicating that significant changes in resist sensitivity occurs following delay. While not wanting to be limited by theory, the processing delay in air may result in reactions with atmospheric species that interact with the photoresist to drive reactions that lead to insolubility of the resist and thus a tendency to lower contrast. The results suggest that in the absence of additives processing delays can lead to significant and uncontrolled changes in photoresist performance.

A comparison of the DtS for the film samples prepared with precursor solution CE (absent additives) with the DtS of the additive compositions under the same PEB conditions shows that the additives increased the dose. However, the results also show that there was a lower change in dose after delay when the additives were present. For all additive compositions tested (prepared with photoresist solution samples A1-D3), the magnitude of the change in DtS due to the processing delay was lower than for the photoresists prepared without an additive, ranging from a magnitude of 0.31% to 10.3%. The change in DtS was observed to be further suppressed by increasing the temperature of the PEB from 180° C. to 200° C. or 210° C. The results show that by including an additive in the organotin photoresist composition, changes in patterning performance due to processing delays in ambient environments can be lessened. Given the boiling points of the additives used in this example, it is believed that the additives remain in the photoresist coatings after the PEB and serve to mitigate reactions between the photoresist coating solutions and the photoresist coating with dissolved and atmospheric oxygen and NOx and ROS contaminants.

This example demonstrates the use of additives to effectively lower the dose change associated with post-exposure processing delays. Thus, additives can be used to increase the stability, shelf-life, and batch-to-batch reproducibility of organometallic photoresist compositions.

### Example 3: Patterning Performance Improvements with Radical Scavenger Additives

This example demonstrates the effectiveness of radical scavenger additives in mitigating ambient air contaminant-induced variations in patterning performance. This example also demonstrates the effectiveness of using an inert headspace atmosphere for bottled organotin precursor solutions.

### Preparation of Organotin Precursor Solutions with Radical Scavenger Additives

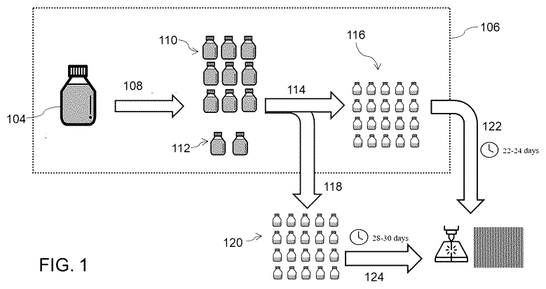

A series of organotin photoresist precursor solutions were prepared. Referring to FIG. 1, stock solution 104 of organotin photoresist precursor solution comprising a blend of organotin trialkoxide compounds having a total tin concentration of 0.055 M [Sn] was formulated in glovebox 106, which was rendered inert with argon. In preparation step 108, photoresist precursor solution samples were prepared. Photoresist precursor solution samples 110 having radical scavenger additives (photoresist precursor solution samples A-I) were prepared by adding a 350 mL aliquot of stock solution 104 to each of nine clean glass bottles and then adding appropriate amounts of radical scavenger agents to each bottle according to Table 5. Photoresist precursor solution sample 112 (photoresist precursor solution sample CTRL), having no radical scavenger agents, was prepared by adding aliquots of stock solution 104 to additional clean glass bottles.

TABLE 5

| Photoresist | | |
|---|---|---|
| Precursor Solution | | Molar Ratio |
| Sample | Radical Scavenger Additive | (Additive:Sn) |
| A | 2,4,6-Tri-tert-butylanaline (TBA) | 0.01:1 |
| B | 1,3,5-Tri-tert-butylbenzene (TTB) | 0.01:1 |
| C | mesitylene | 0.01:1 |
| D | 1,3,5-Trimethoxybenzene | 0.01:1 |
| E | 2,2,6,6-Tetramethylpiperidine | 0.01:1 |
| | (TMP) | |
| F | 2,2,6,6-Tetramethylpiperidine-1- | 0.01:1 |
| | oxyl (TEMPO) | |
| G | butylated hydroxytoluene (BHT) | 0.0001:1 |
| H | butylated hydroxytoluene (BHT) | 0.001:1 |
| I | butylated hydroxytoluene (BHT) | 0.01:1 |
| CTRL | N/A | N/A |

### Preparation of Bottled Organotin Photoresist Samples with Inert and Ambient Headspace

Further referring to FIG. 1, in argon-atmosphere decanting step 114, a 50 mL aliquot of each of photoresist solution samples 110 or photoresist precursor sample 112 was decanted into separate 100 mL bottles within the argon atmosphere of glovebox 106. The bottles were sealed to create photoresist solution samples 116, having an argon headspace atmosphere. Each bottle contained 50 mL of argon headspace and 50 mL of photoresist precursor solution. Photoresist precursor solution samples 116 were stored 122 on the sealed bottles for 22 to 24 days prior to deposition.

In ambient air-atmosphere decanting step 118, a 50 mL aliquot of each photoresist solution samples 110 or photoresist precursor sample 112 was decanted into separate 100 mL bottles in an ambient air atmosphere (outside of glovebox 106). The bottles were sealed to create photoresist precursor solution samples 120, having an ambient air headspace atmosphere. Each bottle contained 50 mL of ambient air headspace and 50 mL of photoresist precursor solution. Photoresist precursor solution samples 120 were stored 124 in the sealed bottles for 28 to 30 days prior to deposition.

Organotin photoresist film samples were then prepared by spin-coating each of the photoresist solution samples onto separate 300 mm Si wafers coated with an organic bottom anti reflection coating (BARC) to produce films having average thicknesses of about 22 to about 24 nm, confirmed using ellipsometry. Following deposition, the film samples were subjected to a post-apply bake (PAB) of 100° C. for 60 seconds. Line-space patterns having a target critical dimension (CD) of 100 nm on a 200 nm pitch were produced for each film sample by exposing the samples to KrF radiation at varying doses between about 80 mJ/cm² and about 270 mJ/cm² to create an array of patterns within fields on the wafer, wherein each field corresponds to the mask pattern printed at a specific dose. As is customary in the art, this type of exposure is referred to as a dose meander exposure. By exposing the same pattern at different doses across each photoresist film sample, the dose required for printing the desired 100 nm lines for a given photoresist film sample (i.e., the dose-to-size for printing 100 nm lines on a 200 nm pitch) can be determined through inspection of each field after processing is complete. Following KrF exposure, each of the film samples were immediately processed by performing a post-exposure bake (PEB) at 180° C. Then development was conducted on each film sample with 2-heptanone and finally each film sample was subjected to a hard bake at 250° C. for 60 seconds to form developed negative tone images.

After the hardbake, the film samples were inspected using a Hitachi CD-SEM to determine the necessary dose to produce patterned lines with a CD of 100 nm, i.e., the dose-to-size for 100 nm lines on a 200 nm pitch. The DtS values for both the film samples prepared from photoresist solution samples stored with argon headspace and the film samples prepared from photoresist solution samples stored with ambient air headspace were collected, as shown in Table 6. Table 6 also records the calculated percent change in DtS between film samples prepared from the differently stored photoresist solution samples. The DtS change shows the efficacy of the radical scavenger in mitigating changes in DtS due to storage conditions in which the photoresist solution sample is exposed to air. A DtS change of 0 would indicate that the storage headspace atmosphere had no impact on the patterning dose needed to achieve 100p200 line-space patterns. A negative DtS change would indicate that the photoresist precursor solution sample stored under air headspace required less dose to achieve the target CD, and a positive DtS change would indicate that the photoresist solution sample stored under air headspace required more dose to achieve the target CD.

TABLE 6

| Photoresist | DtS after ambient | | |
|---|---|---|---|
| Precursor | air headspace | DtS after argon | DtS Change, |
| Solution | storage | headspace storage | (air-argon) |
| Sample | (mJ/cm²) | (mJ/cm²) | % |
| A | 141 | 137 | 2.92% |
| B | 72 | 113 | −36.3% |
| C | 58 | 104 | −44.2% |
| D | 69 | 109 | −36.7% |
| E | 62 | 107 | −42.1% |
| F | 115 | 116 | −0.86% |
| G | 114 | 119 | −4.20% |
| H | 116 | 113 | 2.65% |
| I | 143 | 143 | 0.00% |
| CTRL | 45 | 107 | −57.9% |

The photoresist film sample without radical scavenger additives (prepared from photoresist solution sample CTRL) exhibited the largest change in DtS: −57.9%. This result suggests that storage of an organotin photoresist precursor solution without a radical scavenging additive under an ambient air headspace can lead to an increase of resist sensitivity as well as variations in patterning performance, both of which are undesirable. While not wanting to be limited by theory, the DtS change for the film samples prepared with the control photoresist solution samples may be indicative of reactions between species in the ambient air and species in the photoresist precursor solution since the presence of the radical scavenger additive generally decreased the DtS changes even for the photoresist precursor solutions stored under argon.

The photoresist film samples with radical scavenger additives (prepared from photoresist solution samples A-I) exhibited changes in DtS ranging from 2.92% to −44.2%. These results indicate that the addition of the radical scavenger compounds can effectively mitigate undesirable interactions with ambient air and accordingly reduce the variability in patterning performance of photoresist solutions exposed to air. Positive values for the DtS change were reported for film samples prepared from photoresist precursor solution samples A and H having TBA and BHT radical scavenger additives, respectively. Film samples prepared from photoresist precursor solution sample F (TEMPO additive) and photoresist precursor solution samples G and I (BHT additive) showed from no change in DtS to −4.20% change. These results indicate that TBA, BHT, and TEMPO may be particularly effective radical scavenging compounds for organotin photoresist precursor solutions.

This example demonstrates the use of inert headspace atmospheres above organotin photoresist precursor solutions as well as radical scavenging additives to effectively lower the dose change associated with storage. Thus, the use of radical scavenging additives can be used to effectively mitigate undesirable interactions with ambient air and accordingly reduce the variability in patterning performance of photoresist precursor solutions exposed to air.

### Further Inventive Concepts

A1. A structure comprising a substrate having a surface and a radiation patternable coating along the substrate surface comprising a radiation sensitive metal oxide based, organometallic composition and a radical scavenging additive.

A2. The structure of inventive concept A1 wherein the radical scavenger additive comprises an H-atom donating compound.

A3. The structure of inventive concept A1 wherein the radical scavenger additive comprises a branched alkene, a compound having conjugated unsaturated bonds, a phenolic compound, a hindered amine, a polyketone, a compound having a sterically hindered aminoxyl group, an unsaturated heterocycle, a cyclic diene or derivatives thereof, a hydroxylamine, an organophosphine, a phosphite ester, or an aryl azo compound, or a combination thereof.

A4. The structure of inventive concept A1 wherein the radical scavenger additive is selected from the group consisting of a hindered phenol, a hindered amine, and alkoxyphenol and derivatives thereof.

A5. The structure of inventive concept A1 wherein the radical scavenger additive comprises tertbutyl phenol; butylated hydroxy toluene; (2,2,6,6-Tetramethylpiperidin-1-yl)oxyl; 2,2,6,6-Tetramethylpiperidine; 4-Hydroxy-2,2,6,6-tetramethylpiperidine-1-oxyl; 2-tert-butyl-1,4-benzoquinone; 1,3,5-Trimethoxybenzene; mesitylene; 1,3,5-Tri-tert-butylbenzene; or 2,4,6-Tri-tert-butylanaline; or a combination thereof.

A6. The structure of inventive concept A1 wherein the radical scavenger additive comprises butylated hydroxy toluene; (2,2,6,6-Tetramethylpiperidin-1-yl)oxyl; or guaiacol or a derivative thereof.

A7. The structure of inventive concept A1 wherein the radiation patternable coating comprises a mixture of the organometallic composition and the radical scavenging additive.

A8. The structure of inventive concept A1 wherein the radiation patternable coating comprises one or more organometallic layers comprising the organometallic composition and one or more additive layers comprising the radical scavenging additive.

A9. The structure of inventive concept A1 wherein the organometallic composition comprises Sn.

A10. The structure of inventive concept A9 wherein the molar ratio of the radical scavenger additive to Sn is from about 0.0001 to about 0.25.

All. The structure of inventive concept A1 wherein the organometallic composition comprises Sn—C, Sn—O—Sn, and Sn—OH bonds.

A12. The structure of inventive concept A1 wherein the organometallic composition comprises a monoorganotin material forming an oxo-hydroxo network.

A13. The structure of inventive concept A1 wherein the organometallic composition, when fully hydrolyzed, can be represented by the formula RSnO_{(1.5-(x/2))}(OH)ₓ where 0<x≤3, wherein R is a substituted or unsubstituted hydrocarbyl ligand with 1 to 31 carbon atoms and an Sn—C bond.

A14. The structure of inventive concept A13 wherein R comprises a cyclic alkyl group, an aromatic group, a fluorinated group, an unbranched alkyl group, a branched alkyl group, or a combination thereof.

A15. The structure of inventive concept A13 wherein R comprises a t-butyl group, an iso-propyl group, or a combination thereof.

A16. The structure of inventive concept A13 wherein R comprises a hydrocarbyl ligand substituted with heteroatoms.

A17. The structure of inventive concept A13 wherein the Sn—C bond is radiation sensitive.

A18. The structure of inventive concept A1 wherein the radiation patternable coating is sensitive to EUV radiation at a dose from about 1 mJ/cm² to about 150 mJ/cm².

A19. The structure of inventive concept A1 wherein the radiation patternable coating has an average thickness from about 2 nm to about 40 nm.

A20. The structure of inventive concept A1 wherein the substrate comprises a silicon wafer.

B1. A method of forming a radiation patternable metal oxide-based coating on a substrate surface, the method comprising:
exposing the substrate surface to an organometallic precursor composition with hydrolysable ligands;
exposing the substrate surface to vapor of a radical scavenging additive, wherein exposure to the radical scavenging additive is before, during, after or any combination thereof with respect to exposure to the organometallic precursor composition; and
   forming a radiation sensitive organometallic film associated with the radical scavenging additive.

B2. The method of inventive concept B1 wherein the exposing comprises chemical vapor deposition (CVD), physical vapor deposition (PVD), atomic layer deposition (ALD), or combinations thereof.

B3. The method of inventive concept B1 wherein the exposing the substrate surface to an organometallic precursor composition with hydrolysable ligands is performed in an atmosphere with water vapor.

B4. The method of inventive concept B1 further comprising exposing the radiation sensitive organometallic film to water vapor.

B5. The method of inventive concept B1 wherein exposure to the radical scavenging additive is during exposure to the organometallic precursor composition.

B6. The method of inventive concept B1 wherein the radiation sensitive organometallic film comprises a mixture of an organotin oxo-hydroxo composition and the radical scavenging additive.

B7. The method of inventive concept B1 wherein the radiation sensitive organometallic film comprises one or more organometallic layers comprising an organotin oxo-hydroxo composition and one or more additive layers comprising the radical scavenging additive.

B8. The method of inventive concept B1 wherein the radical scavenger additive comprises an organophosphate.

B9. The method of inventive concept B1 wherein the organometallic precursor composition comprises an organotin compound represented by the formula RSnL₃ wherein R is a substituted or unsubstituted hydrocarbyl ligand with 1 to 31 carbon atoms and an Sn—C bond and L is a hydrolysable metal ligand.

B10. The method of inventive concept B1 further comprising heating the radiation sensitive organometallic film, wherein the radical scavenging additive is retained.

B11. The method of inventive concept B1 further comprising heating the radiation sensitive organometallic film to remove the radical scavenging additive.

B12. The method of inventive concept B1 wherein the radiation sensitive film exhibits a change of dose-to-size that changes by no more than about 5% as a result of storage or delays allowing exposure to radicals and related reactive species.

B13. The method of any one of inventive concepts B1 to B12 wherein the formation of the radiation sensitive film forms a structure of any one of inventive concepts A1 to A20, wherein the radiation sensitive organometallic film is the radiation patternable coating.

C1. A method of producing a container of radiation sensitive organometallic precursor composition, the method comprising:
forming a stabilized solution comprising dissolved organometallic precursor composition and a dissolved selected quantity of radical scavenging additive; and
sealing the stabilized organometallic precursor solution in a container for distribution.

C2. The method of inventive concept C1 wherein the radical scavenger additive comprises an H-atom donating compound.

C3. The method of inventive concept C1 wherein the stabilized organometallic precursor solution comprises from about 0.000025M to about 0.4M of the radical scavenger additive.

C4. The method of inventive concept C1 wherein the stabilized organometallic precursor solution comprises Sn ions in a concentration from about 0.0025M to about 1 M.

C5. The method of inventive concept C1 wherein the solution comprises an organotin compound represented by the formula RSnL₃ wherein R is a substituted or unsubstituted hydrocarbyl ligand with 1 to 31 carbon atoms and an Sn—C bond and L is a hydrolysable metal ligand.

C6. The method of inventive concept C1 wherein the container is a glass bottle.

C7. The method of inventive concept C6 wherein the container is sealed with an inert atmosphere over the solution.

C8. The method of any one of inventive concepts C1 to C7 wherein the stabilized organometallic precursor solution comprises the precursor solution of any one of claims 1 to 29.

In the above disclosure, it should be understood that certain terms are used interchangeably with each other. For example, one of ordinary skill in the art will understand that the terms “coating”, “layer”, and “film” are meant to construe the same idea, unless explicitly stated otherwise.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understood that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. As would be understood by a person of ordinary skill in the art, the use of the term “about” herein refers to measurement error for the particular parameter unless explicitly indicated otherwise.

What is claimed is:

1. A precursor solution comprising organic solvent, a radiation sensitive organometallic precursor composition with hydrolysable metal ligands, and a radical scavenger additive.

2. The precursor solution of claim 1 wherein the radical scavenger additive comprises an H-atom donating compound.

3. The precursor solution of claim 1 wherein the radical scavenger additive comprises a branched alkene, a compound having conjugated unsaturated bonds, a hindered phenol, a hindered amine, an aromatic diol, a sterically hindered aminoxyl group, an organophosphine, a phosphite ester, or a combination thereof.

4. The precursor solution of claim 1 wherein the radical scavenger additive is selected from the group consisting of a hindered phenol, a hindered amine, an alkoxylphenol and derivatives thereof.

5. The precursor solution of claim 1 wherein the radical scavenger additive comprises tertbutyl phenol; butylated hydroxy toluene; (2,2,6,6-Tetramethylpiperidin-1-yl)oxyl; 2,2,6,6-Tetramethylpiperidine; 4-Hydroxy-2,2,6,6-tetramethylpiperidine-1-oxyl; 2-Phenyl-4,4,5,5-tetramethylimidazoline-1-oxyl 3-oxide; 2-tert-butyl-1,4-benzoquinone; 1,3,5-Trimethoxybenzene; mesitylene; 1,3,5-Tri-tert-butylbenzene; or 2,4,6-Tri-tert-butylanaline; or a combination thereof.

6. The precursor solution of claim 1 wherein the radical scavenger additive comprises butylated hydroxy toluene; (2,2,6,6-Tetramethylpiperidin-1-yl)oxyl; or guaiacol or a derivative thereof.

7. The precursor solution of claim 1 wherein the radical scavenger additive comprises a hindered phenolic compound represented by Formula I: 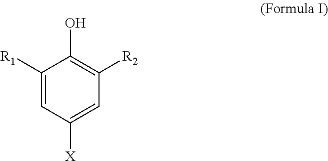
wherein R₁ and R₂ are independently alkyl groups having 1 to 4 carbon atoms and X is selected from —OH, —NH₂, —NMe₂, —OMe, —CH₂Sn(CH₃)₃.

8. The precursor solution of claim 7 wherein R₁ and R₂ are tert-butyl.

9. The precursor solution of claim 1 wherein the radical scavenger additive comprises an alkoxyphenol compound represented by Formula II: 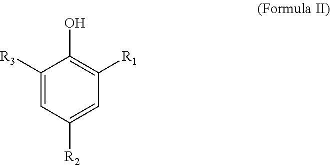
wherein R₁, R₂, and R₃ are independently H or —OR′ where R′ is an alkyl group having from 1 to 4 carbons, and where at least one of R₁, R₂, or R₃ is —OR′.

10. The precursor solution of claim 9 wherein R₁, R₂, and/or R₃ is a methoxy (—OCH₃) group.

11. The precursor solution of claim 1 wherein the radical scavenger additive comprises a hindered amine compound represented by Formula III: 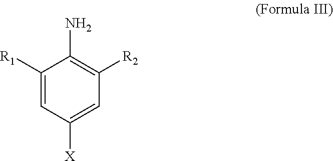
wherein R₁ and R₂ are independently alkyl groups having 1 to 4 carbon atoms and X is H or an alkyl group having 1 to 4 carbon atoms.

12. The precursor solution of claim 11 wherein R₁ and R₂ are tert-butyl.

13. The precursor solution of claim 12 wherein X is tert-butyl.

14. The precursor solution of claim 1 wherein the precursor solution comprises from about 0.000025M to about 0.4M of the radical scavenger additive.

15. The precursor solution of claim 1 wherein the organic solvent comprises an alcohol, an ether, an ester, or a ketone, or mixtures thereof.

16. The precursor solution of claim 1 wherein the organic solvent comprises one or more alcohol.

17. The precursor solution of claim 1 wherein the organic solvent comprises 1-propanol, 1-pentanol, 4-methyl-2-pentanol, or combinations thereof.

18. The precursor solution of claim 1 wherein the radiation sensitive organometallic precursor composition comprises an organotin compound represented by the formula RSnL₃ wherein R is a substituted or unsubstituted hydrocarbyl ligand with 1 to 31 carbon atoms and an Sn—C bond and L is a hydrolysable metal ligand.

19. The precursor solution of claim 18 wherein R comprises a cyclic alkyl group, an aromatic group, a fluorinated group, an unbranched alkyl group, a branched alkyl group, or a combination thereof.

20. The precursor solution of claim 18 wherein R comprises a t-butyl group, an iso-propyl group, or a combination thereof.

21. The precursor solution of claim 18 wherein R comprises a hydrocarbyl ligand substituted with heteroatoms.

22. The precursor solution of claim 18 wherein the Sn—C bond is radiation sensitive.

23. The precursor solution of claim 1 wherein the hydrolysable metal ligand is an alkoxide, a dialkylamide, an alkylacetylide, an alkylsilylamide, or a combination thereof.

24. The precursor solution of claim 1 wherein the hydrolysable metal ligand is tert-butoxide, sec-butoxide, pentan-3-yloxide, tert-amyloxide, or a combination thereof.

25. The precursor solution of claim 18 wherein the molar ratio of the radical scavenger additive to Sn is from about 0.0001 to about 0.25.

26. The precursor solution of claim 1 having a tin concentration from about 0.0025M to about 1 M.

27. The precursor solution of claim 1 having a controlled amount of water.

28. The precursor solution of claim 1 having from about 200 ppm to about 10,000 ppm by weight of water.

29. The precursor solution of claim 1 wherein the precursor solution can be stored under an oxygen-containing atmosphere for at least about 7 days and can be used to form a film that can be radiation pattered with a dose-to-size result within 5% of a dose-to-size relative to an equivalent precursor solution stored under an inert atmosphere.
